(19) United States
(12) Patent Application Publication
(10) Pub. No.: US 2026/0250780 A1
(43) Pub. Date: Aug. 27, 2026

(54) APPARATUS, SYSTEMS, AND METHODS FOR DETECTING INTRA-GENIC VARIANTS AND LOSS-OF-FUNCTION EVENTS

(71) Applicant: SOPHIA GENETICS S.A., Rolle (CH)

(72) Inventors: Fuad MOHAMMAD, Columbus, OH (US); Bernardo Javier FOTH, Montreux (CH); Christian POZZORINI, Lausanne (CH); Alex TUCK, Yverdon-les-Bains (CH); Zhenyu Xu, Nyon (CH)

(73) Assignee: SOPHIA GENETICS S.A., Rolle (CH)

(21) Appl. No.: 19/545,068

(22) Filed: Feb. 20, 2026

Related U.S. Application Data
(60) Provisional application No. 63/761,899, filed on Feb. 22, 2025.

(51) Int. Cl. C12Q 1/6886; C12N 15/10

(52) U.S. Cl. CPC C12Q 1/6886; C12N 15/1065; C12N 15/1089; C12Q 2600/156

(57) ABSTRACT

A method for detecting variants within one or more genes of interest, the method comprising: designing a set of capture probes to cover at least one of the one or more genes of interest, including coding regions and non-coding regions; using the set of capture probes to prepare a sequencing library using a hybrid capture target-enrichment library preparation workflow; and identifying intra-genic structural variants.

### CROSS REFERENCE TO RELATED APPLICATIONS

The present application claims the benefit of U.S. Patent Application No. 63/761,899 for Apparatus, Systems, and Methods for Detecting Intra-Genic Structural Variants, filed on Feb. 22, 2025, the entire contents of which are incorporated herein by reference.

### FIELD OF THE PRESENT DISCLOSURE

The present disclosure is directed to systems and methods for detecting intra-genic variants and loss-of-function events.

### BACKGROUND OF THE INVENTION

Genetic mutations accumulate during the lives of individuals and are ultimately passed through to their offspring. While most mutations do not have a detectable effect on an individual's phenotype, some mutations are pathogenic and can be linked to hereditary diseases if they occurred in the germline. Additionally, some mutations may also be linked to somatic diseases, such as cancer. The nature of the mutation causing a genetic disease can affect the individual's phenotype, as well as the prognosis of the subject and their response to various treatment options. As such, identifying mutations causing genetic diseases, such as cancer, is a cornerstone of many fields of modern medicine.

The advent of next-generation sequencing (NGS; also known as high-throughput sequencing) has drastically reduced the costs of accessing genomic information. The costs associated with sequencing whole genomes of individuals, however, remains prohibitive for most medical applications.

Therefore, it has become common practice to focus sequencing efforts on a part of the genome that has been selectively enriched prior to sequencing, using target enrichment. Typically, target enrichment focuses on a list of genes frequently linked to a particular disease. However, selectively enriching a large list of genes is becoming more common. For instance, enriching a large list of genes may play a role in comprehensive genomic profiling, clinical exome sequencing, or whole exome sequencing panels. Independent of the number of genes being targeted, with few exceptions, target enrichment generally focuses on the coding parts of the genes. Besides providing direct information about mutations altering the encoded protein, coding sequences have the advantage of being more conserved, facilitating the design of probes or primers for target enrichment.

However, sequencing efforts restricted to coding sequences limit the insights to be gained from the data. For example, non-coding mutations might be missed. Second, structural variants might be more difficult to detect. The statistical power to detect copy number variants (CNV) for regions within genes also depends on the number of such regions that are sampled and their size. Thus, CNV analyses restricted to exons are likely to be less powerful, especially for genes having few exons and/or short exons. Similarly, the detection of breakpoints for fusions and other structural rearrangements is less likely when focusing on exclusively exon sequences, as breakpoints occurring in introns would be missed. Therefore, the proportion of missed breakpoints increases for genes with larger proportions of intron sequences compared to exon sequences. In cases where detection of intragenic CNV and structural variants is important, intronic sequences must be included. Added to the importance of relevance of some substitutions and short indels in non-coding regions for various diseases and conditions (Schobers et al. 2024, “Uncovering recessive alleles in rare Mendelian disorders by genome sequencing of 174 individuals with monoallelic pathogenic variants”, European Journal of Human Genetics 33, 56-64), methods for efficiently capturing non-coding sequences without massively increasing the costs of the assays are urgently needed.

Non-coding regions can be included in target-enrichment NGS assays, by designing capture probes (for hybrid capture target enrichment) or PCR primers (for amplicon target enrichment) near or within non-coding sequences. As the amount of non-coding sequence in the genome by far exceeds the amount of coding sequences, there is however a risk of drastically increasing the total length of the gene panel footprint (the total length of genome captured by the probes or amplified by the PCR primers) if non-coding sequences are added without due considerations for their usefulness in follow-up bioinformatic analyses. Whole-genome sequencing (WGS) represents an attractive alternative to target enrichment of non-coding sequences, as the approach sequences most regions of the genome, independently of whether they are coding or non-coding.

However, the total amount of sequencing needed to achieve the same coverage (average number of reads covering each position) drastically increases, leading to important cost hikes. A viable alternative might consist in so-called low-pass WGS, where the whole genome is sequenced at low to moderate coverages (e.g., <1×, 1-5×, or up to 30×). The lower coverage however represents a challenge for the subsequent bioinformatic analyses, again requiring a careful evaluation of the data needed to gain important clinical insights. In addition, many of the tools available to detect genetic variants were developed with an explicit or implicit focus on coding regions, so that new analytical solutions may be required to gain clinically-relevant insights from non-coding regions. There is therefore a need for novel analytical methods to extract clinical insights from data produced using low-pass WGS or target enrichment of non-coding sequences.

The clinical relevance of non-coding regions is already well established for some genes. As an example, approximately 50% of patients with advanced hormone receptor (HR)-positive breast cancer, who typically experience resistance or tumor progression after endocrine-based treatment, harbor mutations in the PIK3CA/PTEN/ATK1 pathway (Turner et al. 2021, “Ipatasertib plus paclitaxel for PIK3CA/AKT1/PTEN-altered hormone receptor-positive HER2-negative advanced breast cancer: primary results from cohort B of the IPATunity 130 randomized phase 3 trial”, Breast Cancer Research and Treatment 191, 565-576; Bhave et al. 2024, “Comprehensive genomic profiling of ESR1, PIK3CA, AKT1, and PTEN in HR(+)HER2(−) metastatic breast cancer: prevalence along treatment course and predictive value for endocrine therapy resistance in real-world practice”, Breast Cancer Research and Treatment 207, 599-609).

These patients may be eligible for targeted treatments, such as PIK3CA/AKT1 inhibitors (Fonucci et al. 2024, “Practical treatment strategies and novel therapies in the phosphoinositide 3-kinase (PI3K)/protein kinase B (AKT)/mammalian target of rapamycin (mTOR) pathway in hormone receptor-positive/human epidermal growth factor receptor 2 (HER2)-negative (HR+/HER2−) advanced breast cancer”, ESMO Open 9, 103997), making it essential to identify these mutations. However, current NGS technologies present challenges when attempting to detect mutations.

This is especially true when diverse substitutions and structural alterations can inactivate PTEN, rendering loss-of-function mutations difficult to detect based solely on exonic sequences. As such, providing clinically relevant variant information from genes such as PTEN requires sequencing of intronic sequences, followed by analyses using dedicated tools to detect intra-genic structural variants with precisions, alongside an accurate detection of mutations in coding sequences and the incorporation of different lines of evidence into gene-level assessments.

Accordingly, it would be desirable to provide systems and methods that sequence intronic and exonic parts of genes that are of interest using NGS workflows that remain affordable by limiting the total amount of sequence data required. Further, it would be desirable to provide systems and methods that analyze data to infer intra-genic gene copy number variants and other structural variants, and to combine different lines of evidence to infer loss of function of genes of interest. It would be yet further desirable to provide systems and methods to design probes tiling whole genes to facilitate efficient target enrichment of intronic regions of genes of interest, wherein the inclusion of said regions increases the information available for calling CNVs and other intra-genic structural variants, and increases analytical accuracy, especially for genes with few and/or short exons.

### SUMMARY OF THE INVENTION

This summary is provided to introduce a selection of concepts in a simplified form that are further described below in the detailed description. This summary is not intended to identify key features or essential features, nor is it intended to limit the scope of the claims included herewith.

Aspects of the present disclosure are directed to a method to detect loss of function variants within one or more genes of interest in a subject suffering from, or suspected to suffer from, a cancer, the method comprising the steps of: (i) generating a whole-genome sequencing library from a plurality of DNA fragments derived from genomic DNA extracted from cells or cell-free DNA derived from bodily fluids; (ii) using high-throughput sequencing to obtain sequencing reads from the sequencing library; (iii) subjecting the sequencing reads to quality-based filters and mapping the resulting sequencing reads onto a reference genome; (iv) detecting, from the sequencing reads, genetic variants that belong to at least two distinct categories, such as single nucleotide variants (SNVs), short insertions/deletions (indels), intra-genic copy number variants (CNV), large copy number variants (CNVs), and structural rearrangements; and (v) combining information from at least two different types of genetic variants to determine whether the subject comprises loss of function of the one or more genes of interest.

In some embodiments, the whole-genome sequencing library is subjected to hybrid-capture target enrichment to produce a capture library prior to sequencing, and wherein capture probes cover coding and non-coding regions of the one or more genes of interest.

In some embodiments, part of the whole-genome sequencing library is mixed with the capture library before sequencing, and wherein the concentrations of the whole-genome sequencing library and capture library in the mix are selected to obtain a lower coverage for the whole-genome sequencing library in the sequencing reads.

In some embodiments, wherein the reads originating from the capture library are used to detect intra-genic variants, such as SNVs, indels, CNVs, and structural variants.

In some embodiments, structural variants are identified based on the presence of discordant gene pairs or reads spanning sequences that are not contiguous within the reference genome.

In some embodiments, algorithms are deployed to detect discordant gene pairs or reads spanning non-contiguous reference sequences in numbers that allow confident inference.

In some embodiments, CNVs are inferred from coverage data, corresponding to the number of reads covering each position.

In some embodiments, inferring the CNVs is performed using a hidden Markov model (HMM), which assigns copy numbers based on observed coverage data and an underlying model.

In some embodiments, CNVs are inferred independently, at the whole-gene level and at the exon level, and wherein the whole-gene level and exon level CNV estimates are combined to report CNVs within genes in addition to CNV of the whole gene.

In some embodiments, the exon-level CNVs are inferred based on the coverage of genuine exons and virtual exons defined within the introns.

In some embodiments, estimates of gene-level CNVs and exon-level CNVs are combined with read-based estimates of structural variants to infer rearrangements, by reporting high-confidence structural variants when they are detected with both methods.

In some embodiments, the reads originating from the whole-genome sequencing library are used to calculate coverage among genomic bins and infer CNVs among genomic regions spanning at least 10,000 bp.

In some embodiments, the inferring copy number is performed by comparing the coverage of one or more selected genomic bins of interest to other genomic bins along the same chromosome.

In some embodiments, a loss of function is determined if: (i) any of a gene loss, an exon loss, a gene fusion, a SNV causing a premature STOP codon, or an indel causing a reading frame shift is detected; (ii) a SNV that causes an amino acid change that is predicted to be pathogenic is detected; or (iii) a combination thereof.

In some embodiments, homozygous loss of function is inferred if two loss-of-function causing genetic variants can be assigned to distinct alleles.

In some embodiments, the capture probes are designed based on a reference genome, so that they cover most of the gene sequence, including one or more of exons, introns, and UTRs.

In some embodiments, the set of capture probes represent a combination of different sets of probes.

In some embodiments, the one or more genes of interest include the gene PTEN.

Some aspects of the present disclosure describe a computer-implemented software to identify loss-of-function variants from high-throughput sequencing data using the method described above.

Some aspects of the present disclosure describe a set of reagents used to produce sequencing libraries compatible with the method described above.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is a schematic representation of the PI3K/AKT/mTOR pathway (taken from Porta, C. et al Front. Oncol., 13 Apr. 2014).

FIG. 2 illustrates a non-limiting workflow to implement the disclosed method.

FIGS. 3A-3B illustrates exon-level CNV inference for a gene. FIG. 3A shows exon-level CNV inference based solely on real exons. FIG. 3B shows exon-level CNV inference based on both real and virtual exons.

FIG. 4 illustrates an embodiment of an environment in which the systems and methods of the present disclosure may be practiced.

FIG. 5 illustrates an embodiment of a block diagram of an electronic device.

FIG. 6 illustrates an example of PTEN regions for intra-genic analysis of copy number variants.

FIG. 7 illustrates an example of the results from CNV analyses based only on genuine exons of PTEN or based on both genuine and virtual exons.

FIG. 8 shows the distribution of the relative PTEN coverage for clinical samples with independently assessed PTEN status.

FIG. 9 shows coverage plots based on capture reads and WGS reads used to help establish PTEN deletion status.

FIG. 10 shows whole genome sequencing (WGS)-based coverage plots to assess PTEN deletion status.

FIG. 11 illustrates methods to combine WGS and capture data to infer PTEN loss of function.

### DETAILED DESCRIPTION OF THE INVENTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The proposed methods and systems will now be described by reference to more detailed embodiments. The proposed methods and systems may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Disclosed herein may be systems and methods for designing a device to detect variants within genes of interest, systems, and methods for using such a device, and/or an apparatus to detect said variants. In a preferred embodiment, the present disclosure may facilitate improved detection of intra-genic structural variants, including exon gains and losses, fusions, and the like. In another embodiment, the present disclosure may be best suited for genes with large proportions of non-coding to coding sequences. The aforementioned apparatus for detecting variants may use hybrid-capture target enrichment within an NGS workflow, low-pass whole-genome sequencing in an NGS workflow, or a combination of hybrid-capture target enrichment and low-pass WGS.

### Definitions

As used herein, an “adapter” or “adaptor” refers to a short double-stranded or partially double-stranded DNA molecule which has been designed to be ligated to a DNA fragment. An adaptor may have blunt ends, sticky ends as a 3′ or a 5′ overhang, or a combination thereof. For example, to improve ligation efficiency, an adenine may be added to each of the 3′ blunt ends of the fragmented DNA prior to adaptor ligation, and the adaptor may have a thymidine overhang on the 3′ end to base-pair with the adenine added to the 3′ end of the fragmented DNA. The adaptor may have a phosphorothioate bond before the terminal thymidine on the 3′ end to prevent an exonuclease from trimming the thymidine, thus creating a blunt end when the end of the adaptor being ligated is double-stranded.

As used herein, the terms “aligning” or “alignment” or “aligner” refer to mapping and aligning base-by-base, in a bioinformatics workflow, the pre-processed sequencing reads to a reference genome sequence, which may vary among applications. For instance, in a targeted enrichment application where the sequencing reads are expected to map to a specific targeted genomic region in accordance with the hybrid capture probes used in the experimental amplification process, the alignment may be specifically searched relative to the corresponding sequence, defined by genomic coordinates such as the chromosome number, the start position and the end position in a reference genome. The alignment may however also be performed against the whole reference genome, with subsequent filtering out of reads mapping to regions not targeted by the capture panel. The alignment may include a local refinement of the initial alignment to match insertions and deletions inferred within a given genomic window.

As used herein, the terms “AKT1” or “AKT” refer to the gene that encodes AKT serine/threonine kinase, also known as protein kinase B (PKB). AKT is a crucial enzyme that regulates cell survival, proliferation, metabolism, and growth. As a key component of the PI3K/AKT/mTOR pathway, activated AKT promotes cell growth and inhibits apoptosis, making it a crucial driver of cancer when overexpressed or mutated. AKT is activated by phosphorylation (specifically at sites Thr308 and Ser473) in response to growth factors. AKT is an oncogene. Mutations, such as E17K, make it overactive, contributing to various cancers.

As used herein, the term “amplification” refers to a polynucleotide amplification reaction to produce multiple polynucleotide sequences replicated from one or more parent sequences. Amplification may be produced by various methods, for instance a polymerase chain reaction (PCR), a linear polymerase chain reaction, a nucleic acid sequence-based amplification, a rolling circle amplification, and other methods.

As used herein, the term “ATM” refers to the gene that encodes the ataxia-telangiectasia mutated protein. The ATM gene encodes a crucial protein that acts as a master controller for detecting and repairing damaged DNA. Mutations in this gene inhibit DNA repair, significantly increasing cancer risk. The ATM protein identifies broken DNA strands and activates enzymes to repair them, maintaining cellular stability.

As used herein, the term “apoptosis” refers to the cells natural, controlled process of “programmed cell death” or “cellular suicide” used to remove damaged, old, or unnecessary cells. Biochemical events lead to characteristic cell changes (morphology) and death. These changes include blebbing, cell shrinkage, nuclear fragmentation, chromatin condensation, DNA fragmentation, and mRNA decay. The average adult human loses 50 to 70 billion cells each day to apoptosis. In contrast to necrosis, which is a form of traumatic cell death that results from acute cellular injury, apoptosis is a highly regulated and controlled process that confers advantages during an organism's life cycle. Because apoptosis cannot stop once it has begun, it is a highly regulated process. Apoptosis can be initiated through one of two pathways. In the intrinsic pathway, the cell kills itself because it senses cell stress, while in the extrinsic pathway, the cell kills itself because of signals from other cells. Weak external signals may also activate the intrinsic pathway of apoptosis. Both pathways induce cell death by activating caspases, which are proteases, or enzymes that degrade proteins. The two pathways both activate initiator caspases, which then activate executioner caspases, which then kill the cell by degrading proteins indiscriminately. In addition to its importance as a biological phenomenon, defective apoptotic processes have been implicated in a wide variety of diseases. Excessive apoptosis causes atrophy, whereas an insufficient amount results in uncontrolled cell proliferation, such as cancer. Some factors, like Fas receptors and caspases, promote apoptosis, while some members of the Bcl-2 family of proteins inhibit apoptosis.

As used herein, the term “BARD1” refers to the gene that encodes BRCA-1 associated RING domain 1, which acts as a crucial tumor suppressor by binding with the BRCA1 protein to repair damaged DNA and maintain genomic stability. Mutations in BARD1 impair its function, significantly increasing the risk of developing cancer. The BARD1 gene encodes a protein that pairs with BRCA1, creating a complex essential for DNA damage repair and acts as an E3 ubiquitin ligase. Additionally, tumors containing BARD1 mutations may respond to PARP inhibitors.

As used herein, the term “BRCA1” refers to a gene that encodes a tumor suppressor that repairs damaged DNA and prevents cells from growing too rapidly. Mutations in BRCA1 significantly increase the risk of developing cancer. Normally, BRCA1 encodes a protein that repairs DNA damage. When mutated, its function is lost, leading to an accumulation of genetic errors and uncontrolled cell growth.

As used herein, the term “BRCA2” refers to a gene that encodes a tumor suppressor that repairs damaged DNA and prevents cells from growing too rapidly. Mutations in BRCA2 significantly increase the risk of developing cancer. Normally, like BRCA1, BRCA2 encodes a protein that repairs DNA damage. When mutated, its function is lost, leading to an accumulation of genetic errors and uncontrolled cell growth.

As used herein, the term “BRIP1” refers to a gene that encodes the BRCA1-interacting protein C-terminal helicase 1 protein. The BRIP1 gene is a tumor suppressor gene that plays a crucial role in DNA repair. Pathogenic mutations in BRIP1 are associated with increased risk of developing cancer. The BRIP1 protein acts as a helicase, interacting directly with BRCA1 to repair DNA double-strand breaks.

As used herein, the term “CCNE1” refers to a gene that encodes the cyclin E1 protein, which is a critical protein regulating the G1 to S phase transition of the cell cycle by binding to and activating CDK2. CCNE1 is critical for DNA replication, but its amplification and overexpression is frequently associated with increased malignancy, chromosomal instability, and poor prognosis in various cancers. The CCNE1 protein accumulates at the G1/S boundary, promoting cell cycle progression, and is degraded as cells enter S phase. It plays a crucial role in initiating DNA duplication and, when overexpressed, can lead to uncontrolled cell proliferation. CCNE1 functions as a regulatory subunit for CDK2, which phosphorylates target proteins to initiate DNA replication. Normally, CCNE1 levels are tightly controlled by the tumor suppressor FBXW7 through ubiquitin-dependent degradation, however this mechanism is often lost in cancers with CCNE1 amplification.

As used herein, the term “CDK12” refers to the gene that encodes the cyclin dependent kinase 12 protein. The CDK12 protein acts as a key regulator of transcription elongation by phosphorylating RNA polymerase II. It is crucial for maintaining genomic stability, regulating DNA repair, and cell cycle progression. Mutations or loss of CDK12 are frequently observed in various cancers. CDK12 is usually associated with cyclin K and phosphorylates the C-terminal domain of RNA polymerase II, promoting the expression of genes involved in DNA damage response.

As used herein the term “CHEK1” refers to the gene that encodes the checkpoint kinase 1 protein, which is a crucial serine/threonine protein kinase responsible for mediating cell cycle arrest, DNA repair, and damage response. It acts as a central mediator, activating in response to DNA damage or unreplicated DNA by integrating signals from ATM/ATR, and is frequently overexpressed in various cancers. The CHEK1 protein regulates the G2/M checkpoint, S-phase, and mitotic entry, ensuring cell cycle arrest (via CDC25A phosphorylation) for DNA repair. It is involved in stabilizing stalled replication forks and promotes homologous recombination.

As used herein the term “CHEK2” refers to the gene that encodes the checkpoint kinase 2 protein, which is a tumor suppressor gene that plays a critical role in DNA repair and cell cycle regulation, helping to maintain genomic stability. Mutations in CHEK2 often impair this function, leading to an increased risk of developing various cancers. The CHEK2 protein is a serine/threonine kinase that functions as a tumor suppressor by regulating the cell cycle and repairing DNA damage, particularly double strand breaks. CHEK2 is activated by ATM kinase, wherein CHEK2 halts cell division, allowing time for DNA repair or inducing apoptosis in damaged cells.

As used herein, the term “consensus sequencing” refers, in a bioinformatics workflow, to grouping sequencing reads into families of reads that may be issued from the same double-stranded DNA fragment and/or the same DNA fragment strand and generating a single sequence, called consensus sequence, that represents the group of reads. The consensus sequence may represent the most frequent sequence among the reads in the family or may be composed, at each position, of the most frequent nucleotide at the position among reads of the family. Other consensus-building methods are possible. Variant calling is then performed by processing the resulting consensus sequences, rather than the totality of reads.

As used herein, the terms “cell-free DNA” and “cfDNA” refer to DNA molecules that circulate in a subject's body and originate from one or more healthy cells and/or from one or more cancer cells. These DNA molecules are found outside cells, in bodily fluids such as blood, whole blood, plasma, serum, urine, cerebrospinal fluid, fecal, saliva, sweat, tears, pleural fluid, pericardial fluid, or peritoneal fluid of a subject, and are fragments of DNA expelled from healthy and/or cancerous cells, e.g., upon apoptosis and lysis of the cellular envelope. The cell-free DNA can be in the form of microvesicles, exosomes, apoptotic bodies, or DNA-protein complexes. The fraction of cfDNA that originates from cancerous cells is sometimes referred to as “circulating tumor DNA” or “ctDNA”.

As used herein, the terms “complementary DNA” or “cDNA” refer to a synthetic DNA reverse transcribed from RNA through the action of a reverse transcriptase. The cDNA may be single stranded or double stranded and can include strands that have either or both of a sequence that is substantially identical to a part of the RNA sequence or a complement to a part of the RNA sequence.

As used herein, the term “coverage” in reference to NGS refers to the number of reads that align to, or “cover” known reference bases. The coverage may be reported per position or as the average or median over multiple positions. The sequencing coverage level determines whether variant discovery can be made with a certain degree of confidence at particular base positions. Across multiple genomic regions, the average coverage can be computed as the read count, often considering only high-quality reads effectively mapped onto the reference genome, multiplied by the read length and divided by the total length of the considered genomic regions. At a higher level of coverage, each base is covered by a greater number of aligned sequence reads, and mutations at the base level compared to a reference sample can be determined.

As used herein, a “DNA sample” refers to a nucleic acid sample derived from an organism, as may be extracted for instance from a body tissue or fluid. The organism may be a human, an animal, a plant, fungi, or a microorganism. The nucleic acids may be found in limited quantity or low concentration, such as fetal circulating DNA (cfDNA) or circulating tumor DNA in blood or plasma. A DNA sample also applies herein to describe RNA samples that were reverse-transcribed and converted to cDNA.

As used herein, a “DNA fragment” refers to a short piece of DNA resulting from the fragmentation of high molecular weight DNA. Fragmentation may have occurred naturally in the sample organism, or may have been produced artificially from a DNA fragmenting method applied to a DNA sample, for instance by mechanical shearing, sonification, enzymatic fragmentation and other methods. After fragmentation, the DNA pieces may be end repaired to ensure that each molecule possesses blunt ends. To improve ligation efficiency, an adenine may be added to each of the 3′ blunt ends of the fragmented DNA, enabling DNA fragments to be ligated to adaptors with complementary dT-overhangs.

As used herein, a “DNA product” refers to an engineered piece of DNA resulting from manipulating, extending, ligating, duplicating, amplifying, copying, editing and/or cutting a DNA fragment to adapt it to a next-generation sequencing platform.

As used herein, a “DNA-adaptor product” refers to a DNA product resulting from ligating a DNA fragment with a DNA adaptor to adapt it to a next-generation sequencing workflow. The DNA adaptor may contain one or more primer-binding sites, one or more sample barcodes (also known as indexes), one or more molecular identifiers, or other sequences useful for downstream analyses.

As used herein, a “DNA library” refers to a collection of DNA products or DNA-adaptor products to adapt DNA fragments for compatibility with a next-generation sequencing platform.

The term “enrich” as used herein refers to increasing the proportion of a desired substance, for example, to increase the relative frequency of at least one nucleic acid sequence compared to its natural frequency in a high throughput sequencing reaction. Positive selection, negative selection, or both are generally considered necessary to any enrichment scheme. Enrichment methods include, without limitation, hybrid-capture enrichment and amplification-based enrichment.

As used herein, the term “exon” refers to a the part of a gene that will form a part of the final mature messenger RNA (mRNA) produced by that gene after introns have been removed by RNA splicing. The term “genuine exon”, as used herein, refers to a biological exon, which is included in at least some of the mature mRNA generated from the gene. The term “virtual exon”, as used herein, refers to a region of a gene or genomic region that is not biologically an exon included in at least some of the mature mRNA, but is considered as an exon for the purpose of some bioinformatic analyses.

As used herein, the term “ESR1” refers to the gene that encodes the estrogen receptor alpha protein, a ligand-activated transcription factor regulating gene expression. Mutations in ESR1 are often implicated in cancer development. ESR1 binds to DNA and recruits co-regulators in response to estrogen, promoting cell growth. Mutations in ESR1 often cause the receptor to remain active, even in the absence of estrogen.

As used herein, the term “FANCA” refers to the gene that encodes a protein essential for the Fanconi anemia (FA) pathway, which repairs DNA interstrand cross-links that block replication. As part of the FA core pathway, FANCA facilitates the monoubiquitination of FANCD2 and FANC1, triggering downstream DNA repair mechanisms. FANCA acts within a complex that senses DNA damage during S phase of the cell cycle. The core complex activates the FANCD2 and FANC1 proteins by attaching ubiquitin molecules, which is a crucial step for activating DNA repair. Mutations in FANCA are often implicated in various cancers.

As used herein, the term “FANCD2” refers to the gene that encodes a protein that is a central effector of the Fanconi anemia pathway, crucial for DNA damage repair, particularly during S-phase when replication forks encounter obstacles like interstrand cross-links. Upon DNA damage, FANCD2 is monoubiquitinated by the FA core complex, enabling it to function as a sliding DNA clamp to stabilize stalled forms, promote homologous recombination, and prevent cell cycle checkpoint failure. In response to replication stress, the FA core complex and UBE2T monoubiquitinate FANCD2. Monoubiquitinated FANCD2 forms a heterodimer for FANC1, which binds to DNA, acting as a sliding clamp that protects DNA and recruits other repair proteins. Mutations in FANCD2 are often implicated in various cancers.

As used herein, the term “FANCL” refers to the gene that encodes a protein that is a crucial E3 ubiquitin ligase that acts as the catalytic core of the FA complex, which is essential for repairing DNA interstrand cross-links. FANCL monoubiquitinates FANCD2 and FANC1, triggering the recruitment of DNA repair machinery during S-phase or DNA damage. FANCL is regulated by phosphorylation and ubiquitin-mediated proteolysis and is influenced by the PI3K/AKT pathway. Mutations have been implicated in various cancers.

As used herein, the term “FGFR1” refers to the gene that encodes the fibroblast growth factor receptor 1 protein, a tyrosine kinase receptor that regulates critical cellular processes like proliferation, differentiation, and migration. It acts by binding FGF ligands, causing receptor dimerization and activating downstream pathways (e.g., MAPK, PI3K, etc.). Mutations are often implicated in cancers. Dimerization triggers transphosphorylation, where each receptor molecule phosphorylates its partner on tyrosine residues. This activates the kinase domain, which then initiates intracellular signaling pathways. Overexpression or genetic can lead to constitutive, or permanent, activation of the FGFR1 pathway. This drives, for example, cell growth and resistance to apoptosis.

As used herein, the term “FGFR2” refers to the gene that encodes a transmembrane receptor tyrosine kinase that regulates cell growth, differentiation, and migration by binding fibroblast growth factors (FGFs). Ligand binding triggers dimerization and phosphorylation of the receptor, activating downstream pathways (e.g., RAS/MAPK, PI3K/AKT). Overexpression, amplification, or gene fusions can render the receptor constantly active, driving tumor growth in various cancers.

As used herein, the term “FGFR3” refers to the gene that encodes the fibroblast growth factor receptor 3 protein, which acts as an oncogene in cancer by driving cell proliferation, survival, and angiogenesis through activating mutations or fusions. These alterations cause constitutive, ligand-independent dimerization and kinase activation of the cell-surface receptor. This, in turn, triggers down-stream signaling pathways like RAS/MAPK, PI3K/AKT, and STAT1/3.

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., a loss or a gain of an exon, gene, or a large span of a chromosome; also called copy number variation; “CNV”), a partial or complete change in the ploidy of the cell, a gene fusion (e.g., a rearrangement leading to parts of two different genes being fused to create a single chimeric gene), a variation in the expression level of a gene, as well as changes in the epigenetic information of a genome, such as altered DNA methylation patterns.

As used herein, a “genomic platform” is a system using at least one computer processor to perform analyses of genomic data, also known as bioinformatic analyses. A genomic platform may take as input files sequencing data, in formats such as binary base call (BCL) or FASTQ. The genomic platform may return lists of variants, various genomic scores, and/or annotation in downloadable files, such as txt, pdf or spreadsheets. The genomic platform may also return results in a user interface or may interact with other software to transfer results. A single genomic platform may be responsible for the entire bioinformatic workflow, but in other settings different genomic platforms may be responsible for distinct parts of the bioinformatic workflow. The genomic platform may be comprised of multiple scripts and software programs, some of which may be custom designed, or the genomic data analyzer may be a stand-alone software. The genomic platform may be locally installed on the user computer or the genomic data analyzer may be cloud based and accessible using a dedicated software or a web browser.

The term “hybridization” refers to the process of combining complementary, single-stranded nucleic acids into a single molecule. Nucleotides will bind to their complement under normal conditions, so two perfectly complementary strands will bind (or ‘anneal’) to each other readily. Nucleotide inconsistencies between the two strands make binding between them more energetically unfavorable. However, hybridization can occur despite inconsistencies and the number of nucleotide differences tolerated for successful hybridization depends on the length of the DNA segment, the GC content of the DNA segment the temperature of the reaction, the salt concentration in the liquid, and the pH of the solution.

The term “intron” as used herein, refers to any nucleotide sequence within a gene that is not expressed or operative in the final mRNA product. Intron sequences are typically spliced out during the maturation of mRNA.

As used herein, the term “ligation” refers to the joining of separate DNA sequences, which may be single-stranded, double-stranded, or partially double-stranded. When double-stranded, the resulting DNA molecules may be blunt ended or may have compatible overhangs to facilitate their ligation. Ligation may be produced by various methods, for instance using a ligase enzyme, performing chemical ligation, and other methods.

As used herein, the term “loss of function” refers to the reduction or elimination of the activity of a gene or gene product (e.g., protein) caused by a mutation or genetic alteration. A loss of function mutation causes the protein to be less active, totally inactive, or not produced at all. Loss of function mutations can prevent protein synthesis, hamper proper folding of the protein, disrupt the enzymatic activity of the protein, create shorter, non-functional proteins, or cause total deletion of the gene.

As used herein, the term “MRE11” refers to the gene that encodes a core component of the MRN DNA-repair complex, acts as a tumor suppressor by detecting and repairing DNA double-strand breaks. Its dual exonuclease/endonuclease functions are crucial for genomic stability. Mutations or loss of MRE11 cause high genomic instability and drive aggressive cancers. Conversely, MRE11 overexpression can promote cancer progression, radio-resistance, and cell proliferation in various tumors. MRE11 forms the MRN complex with RAD50 and NBS1, which acts as a “sensor” for DNA double-strand breaks (DSBs), initiating homologous recombination repair and activating the ATM kinase checkpoint.

As used herein, a “molecular tag” or “molecular barcode” or “molecular code” or “molecular identifier” refers to a molecular arrangement such as a nucleic acid sequence which is fully and uniquely specified by its string of nucleotides. Molecular identifiers can be endogenous, as represented by the position of the nucleic acid sequence with respect to a reference genome, the start and/or end sequences of the DNA nucleic acid sequence, or other properties of the nucleic acid sequences. Molecular identifiers can be exogenous, based on sequences incorporated during the library preparation. Exogenous molecular identifiers may be incorporated as part of a ligated adaptor or as part of primers used for amplification. Exogenous molecular identifiers may consist in a large pool of distinct sequences or a limited number of distinct sequences. Exogenous molecular identifiers may differ from each other based on their nucleotide sequence, their length, or a combination of sequence and length. Unique molecular identifiers can result from endogenous molecular identifiers, exogenous molecular identifiers, or a combination of endogenous and exogenous molecular identifiers, as described in Kivioja et al. 2012, “Counting absolute numbers of molecules using unique molecular identifiers”, Nature Methods 9, 72-74. Unique molecular identifiers can be used to identify reads potentially representing PCR duplicates originating from the same original DNA fragment.

As used herein, the terms “mechanistic target of rapamycin” or “mTOR” refer to a central protein kinase that acts as a nutrient sensor to regulate cell growth, division, metabolism, and survival. By integrating signals like energy levels and growth factors, it controls anabolic processes and is heavily implicated in aging, cancer, and metabolic diseases. mTOR regulates protein synthesis, lipid synthesis, and autophagy. Hyperactive mTOR is associated with cancer, as it drives tumor growth.

As used herein, the terms “NBN” refers to the gene that encodes the nibrin protein that is crucial for repairing DNA double-strand breaks as part of the MRN complex, which maintains genomic stability. Mutations in NBN are linked to increased risk of cancer. The nibrin protein, in complex with MRE11 and RAD50, detects DNA damage and alerts cell cycle checkpoints to repair, or remove, damaged cells. When NBN is mutated, DNA repairs are faulty, causing high-level chromosomal instability. This accumulation of DNA damage allows cells to grow and divide uncontrollably, leading to cancer.

As used herein, the terms “Next Generation Sequencing” or “NGS” or “High-Throughput Sequencing” refer to a method of parallel sequencing. For instance, a nucleic acid (e.g., DNA) sample is obtained and prepared into a library (meaning a collection of nucleic acid fragments from the sample). The library may be prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. According to some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. The term “NGS read length” as used herein refers to the number of base pairs (bp) sequenced from a DNA fragment, or each end of a DNA fragment. After sequencing, the sequencing reads may be aligned onto a reference genome, enabling comparison between the sample and the reference.

As used herein, the terms “nucleotide sequence” or a “polynucleotide sequence” refer to any polymer or oligomer of nucleotides such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string). It may be DNA or RNA, or a combination thereof. It may be found permanently or temporarily in a single-stranded or a double-stranded shape. Unless otherwise indicated, nucleic acids sequences are written left to right in 5′ to 3′ orientation.

As used herein, the term “PALB2” refers to the gene that encodes the partner and localizer of BRCA2 protein that is a crucial tumor suppressor that maintains genome stability by repairing DNA double-strand breaks through homologous recombination. It acts as a scaffold, linking BRCA1 and BRCA2 to facilitate DNA repair. Mutations in PALB2 impair this repair mechanism, causing genomic instability and significantly increasing the risk of developing breast, ovarian, and pancreatic cancers.

As used herein, a “PCR duplicate” refers to a copy generated by PCR amplification from a single stranded DNA molecule belonging to an original DNA fragment or a DNA-adaptor product derived from an original DNA fragment.

As used herein, the term “PI3K” refers to is an enzyme critical for regulating cell growth, proliferation, survival, motility, and metabolism. As a key intracellular signal transducer, it converts PIP2 into PIP3 at the plasma membrane, triggering downstream signaling (i.e., AKT/mTOR), in response to growth factors. Dysregulation of this pathway is heavily involved in cancer and immune diseases. PI3K is a lipid kinase that phosphorylates the 3-position hydroxyl group of the inositol ring of phosphatidylinositol. PI3K is activated by receptor tyrosine kinases (RTKs) or G-protein coupled receptors (GPCRs). Hyperactivation of PI3K is frequently found in various cancers, as it promotes tumor cell survival and growth. The enzyme consists of a regulator subunit and a catalytic subunit.

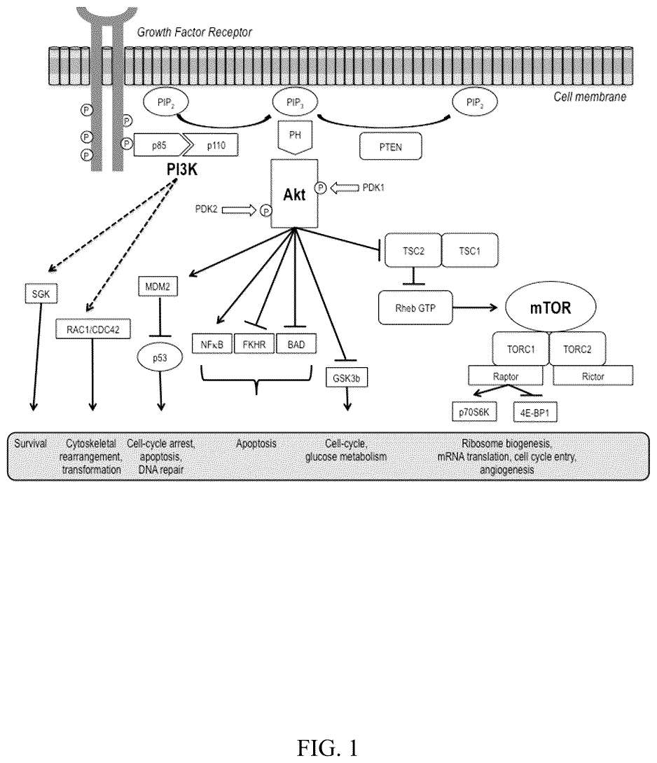

As used herein, the term “PI3K/ATK/mTOR pathway” refers to a central intracellular signaling cascade that links extracellular growth factor stimulation to cellular programs governing proliferation, survival, metabolism, and protein synthesis. A schematic of which is provided as FIG. 1. The pathway is typically initiated when growth factors bind and activate receptor tyrosine kinases at the cell surface, resulting in recruitment and activation of PI3K. Activated PI3K phosphorylates PIP2 (phosphatidylinositol-4,5-biphosphate) to generate PIP3 (phosphatidylinositol-3,4,5-triphosphate), a lipid second messenger that accumulates at the plasma membrane and recruits proteins containing pleckstrin homology domains, most notably AKT. AKT is then phosphorylated and fully activated by PDK1 and mTOR complex 2 (mTORC2). Once activated, AKT phosphorylates numerous downstream substrates that promote cell survival, glucose metabolism, and cell cycle progression. A key downstream effector is mTOR, which functions in two multiprotein complexes. mTOR complex 1 (mTORC1) stimulates protein synthesis and cellular growth through phosphorylation of S6 kinase and 4E-BP1, while mTORC2 contributes to cytoskeletal regulation and reinforces AKT activation. Through these coordinated signaling events, the pathway integrates growth factor availability and nutrient status to drive growth and prevent apoptosis.

As used herein, the term “PIK3CA” refers to the gene that encodes the p110α catalytic subunit of phosphatidylinositol 3-kinase (PI3K).

As used herein, the terms “PIK3CA/PTEN/ATK1 pathway” or “PIK3CA/ATK1/PTEN pathway” or “PI3K/AKT/PTEN pathway” refers to the core upstream regulator of the broader PI3K/ATK/mTOR pathway (a schematic of which is provided as FIG. 1). This is a critical regulator step in the PI3K/ATK/mTOR pathway and is a dynamic balance between PI3K-mediated generation of PIP3 and its removal by the tumor suppressor PTEN. PTEN is a lipid phosphatase that directly antagonizes PI3K by dephosphorylating PIP3 back to PIP2, thereby limiting the membrane recruitment and activation of AKT. In this manner, PTEN functions as the principle negative regulator on PI3K/AKT signaling. When PTEN activity is intact, PIP3 levels are tightly controlled, ensuring that AKT activation is transient and stimulus dependent. However, loss of PTEN function through mutation, deletion, or epigenetic silencing results in accumulation of PIP3, constitutive AKT activation and persistent downstream signaling, even in the absence of upstream growth factor stimulation. Functionally, PTEN loss can phenocopy activating mutations in PI3K because both alterations converge on excessive PIP3 production and sustained AKT signaling. Thus, the PI3K/AKT/PTEN pathway represents a finely tuned regulator circuit in which PI3K drives proliferation and survival signaling, while PTEN restrains this activity to maintain cellular homeostasis.

As used herein, the term “pool” refers to multiple DNA samples (for instance, 8 samples, 48 samples, 96 samples, or more) derived from the same or different organisms, as may be multiplexed into a single high-throughput sequencing analysis or a single step within a high-throughput sequencing workflow. Each sample may be identified in the pool by a unique sample barcode also referred to as sample index.

As used herein, the term “PPP2R2A” refers to the gene that encodes the B55α regulatory subunit of Protein Phosphatase 2A (PP2A). PPP2R2A is a tumor suppressor gene, wherein its dysfunction via deletion or mutation is common in various cancers, causing increased tumor growth, DNA damage resistance, and altered immune responses. Reduced PPP2R2A often triggers cGAS-STING pathways, increases PD-L1 expression, and sensitizes cells to specific inhibitors. PPP2R2A deficiency (or insufficiency) increases cytosolic DNA, activating the cGAS-STING-type I interferon pathway. This upregulates the PD-L1 immune checkpoint protein, creating a “hot” tumor microenvironment with more NK cells and reduced regulatory T cells (Tregs), making tumors more sensitive to immune checkpoint blockade. As a component of PP2A, PPP2R2A regulates centrosome maintenance. Its loss leads to abnormal centrosome numbers, chromosome segregation failure, and accelerated tumor progression. Loss of PPP2R2A impairs homologous recombination (HR). This deficiency increases cancer cell reliance on PARP enzymes for survival.

As used herein, the term “PTEN” refers to the gene that encodes the phosphatase and tensin homolog protein and is a crucial tumor suppressor gene. PTEN regulates cell survival and division by inhibiting the PI3K/AKT signaling pathway, preventing cells from growing or dividing too rapidly. Mutations in PTEN are linked to various cancers. PTEN acts as a brake on cell proliferation. When PTEN is mutated and its function is lost, this brake is removed, leading to uncontrolled cell growth. Beyond controlling cell division, PTEN is involved in apoptosis, cell migration, and maintaining genetic stability. PTEN functions by dephosphorylating PIP3, effectively reversing the action of PI3K, thus blocking the pathway that signals cell growth and proliferation.

As used herein, a “primer sequence” refers to a nucleotide sequence comprising a region of complementarity to a target DNA a part or all of which is to be elongated or amplified. Primer sequences are often more than 20 nucleotides in length (20 bp), although shorter primers are used depending on the applications.

As used herein, the term “probabilistic sequencing” refers, in a bioinformatics workflow, to grouping sequencing reads into families of reads issued from the same double-stranded DNA fragment and/or the same DNA fragment strand and performing variant calling directly on this data, by processing the totality of reads from different families in order to compute the probability of data supporting all the possible genotypes at each genomic position to be analyzed, by comparing the data with a probabilistic model.

As used herein, the term “proliferation” refers to the increase in cell number through the combined processes of cell growth and division. It is a tightly regulated, fundamental biological process necessary for tissue development, maintenance, and wound healing. The cycle involves growth (G1, G2 phases), DNA synthesis (S phase), and division (M phase), often controlled by checkpoints.

As used herein, the term “RAD51B” refers to a gene that encodes a crucial tumor suppressor that function by facilitating homologous recombination. Germline mutations, truncating variants, or loss-of-function in RAD51B cause genomic instability, leading to increased susceptibility to various cancers. As a member of the RAD51 protein family, RAD51B forms a stable heterodimer with RAD51C to repair DNA damage. When this gene is mutated or lost, the cell cannot efficiently repair double-strand breaks, resulting in genomic instability that promotes tumor formation.

As used herein, the term “RAD51C” refers to a gene that encodes a protein essential for repairing DNA double-strand breaks (DSBs) via homologous recombination. Mutations in RAD51C impair this repair mechanism, causing genomic instability, which leads to increased risk of cancer. RAD51C is a member of the RAD51 paralog family, crucial for repairing DNA interstrand cross-links and double strand breaks. When RAD51C is mutated, cells cannot accurately repair DNA, leading to high mutation rates. Defective RAD51C prevents proper HR-mediated repair, causing cells to use error-prone repair pathways, resulting in chromosomal rearrangements and increased cancer susceptibility. RAD51C plays a key role in the DNA damage response and checkpoint function (e.g., activating CHEK2), allowing cells to stop and repair damage. Its loss disrupts this, allowing damaged cells to divide.

As used herein, the term “RAD51D” refers to a gene that encodes a tumor suppressor that plays a crucial role in DNA repair via homologous recombination, essential for maintaining genomic stability. Pathogenic mutations or loss of function in RAD51D prevent proper DNA repair, leading to accumulated genetic errors, increased genomic instability, and a significantly higher risk of various cancers. Under normal conditions, the RAD51D protein works with the RAD51 family to mend DNA double-strand breaks. When a mutation inactivates RAD51D, this repair pathway fails. The cell cannot correctly fix DNA errors during replication. Failure to repair DNA leads to widespread genomic damage, including chromosome fragments, deletions, and aneuploidy, allowing cells to divide uncontrollably and form tumors. Because these tumors are deficient in DNA repair, they are often sensitive to PARP inhibitors, which exploit this vulnerability by preventing the tumor cells from repairing their DNA, causing them to die.

As used herein, the term “RAD54L” refers to a gene that encodes an oncogene that promotes cancer progression by enhancing homologous recombination (HR) repair, allowing cancer cells to survive DNA damage, proliferate rapidly, and develop resistance to therapy. It acts as a key HR factor, frequently overexpressed in various cancers. RAD54L is a chromatin-remodeling protein that helps the RAD51 protein bind to DNA, enabling the repair of double-strand breaks (DSBs). In cancer, this efficient repair mechanism prevents cell death during rapid division, facilitating tumor growth. By repairing DNA damage induced by cancer treatments, high RAD54L expression directly causes resistance to radiation and chemotherapeutic agents (e.g., PARP inhibitors). Elevated RAD54L expression promotes cancer cell cycle progression and proliferation while inhibiting senescence (premature aging of cancer cells), particularly through interactions with genes like p53, p21, and pRB. In some cancers, RAD54L expression is associated with higher tumor mutation burdens (TMB) and microsatellite instability (MSI), which may impact the tumor immune microenvironment.

As used herein, the terms “read trimming” or “read pre-processing” refer, in a bioinformatics workflow, to the filtering out, in the sequencing reads, of a set of nucleotides at the start of the read sequence string, such as for instance the nucleotides corresponding to the adaptor sequences, to extract the real DNA fragment sequence to be analyzed. Read pre-processing may include determining the unique molecular identifiers of the sequencing reads.

As used herein, the term “reverse transcription” or grammatical variations thereof, refers to the process of copying the nucleotide sequence of an RNA molecule into a DNA molecule. Reverse transcription can be done by reacting an RNA template with an RNA-dependent DNA polymerase (also known as a reverse transcriptase) under well-known conditions. A reverse transcriptase is a DNA polymerase that transcribes single-stranded RNA into single stranded DNA. Depending on the polymerase used, the reverse transcriptase can also have RNase H activity for subsequent degradation of the RNA template.

As used herein, a “sample barcode” or “sample index” or “index” is a known nucleotide sequence inserted in the DNA products during the library preparation to differently mark DNA fragments originating from different samples. The sample barcodes may be inserted as part of the ligated adaptors or may be subsequently incorporated as part of PCR primers. The sample barcodes are selected to differ among samples processed as part of the same pool, so that barcodes can be used to recognize and separate the reads corresponding to each sample as part of a step known as demultiplexing.

As used herein, the term “sequencing” refers to reading a sequence of nucleotides as a string. High throughput sequencing (HTS) or next-generation-sequencing (NGS) refers to real time sequencing of multiple sequences in parallel, typically between 50 and a few thousand base pairs. Exemplary NGS technologies include those from Illumina, Ion Torrent Systems, Oxford Nanopore Technologies, Complete Genomics, Pacific Biosciences, and others. Depending on the actual technology, NGS sequencing may require sample preparation with sequencing adaptors or primers to facilitate further sequencing steps, as well as amplification steps so that multiple instances of a single parent molecule are sequenced, for instance with PCR amplification prior to delivery to flow cell in the case of sequencing by synthesis.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp).

As used herein, the term “TP53” refers to a gene that encodes the p53 protein, which acts as a crucial tumor suppressor, which detects cellular stress or DNA damage to trigger repair, cell cycle arrest, or apoptosis. When mutated—occurring in ~50% of human cancers—p53 loses this protective function, allowing damaged cells to proliferate, often gaining new oncogenic, metastasis-promoting functions. In healthy cells, p53 is maintained at low levels. Upon DNA damage, hypoxia, or oncogene activation, p53 stabilizes, binds to specific DNA sequences, and activates genes that arrest the cell cycle (allowing repair) or induce apoptosis (programmed cell death) if the damage is irreparable. TP53 is frequently mutated via missense substitutions, often in the DNA-binding domain, resulting in a protein that cannot bind to DNA to trigger its protective functions. A single mutation (missense) can inactivate the protein, while the mutant protein can also bind to and inactivate any remaining normal p53 (dominant negative effect). Mutant p53 proteins can also acquire “gain-of-function” properties, such as driving genomic instability, promoting tumor cell proliferation, enhancing metabolic changes, and fostering cancer metastasis.

As used herein, the term “untranslated region” or “UTR” refers to specific sections of messenger RNA (mRNA) located directly before the start codon (5′ UTR) and after the stop codon (3′ UTR). While transcribed from DNA, UTRs are not translated into protein, instead acting as crucial regulatory elements that control mRNA stability, localization, and translation efficiency.

As used herein, the terms “variant calling” or “variant caller” or “variant call” refer to identifying, in the bioinformatics workflow, actual variants in the aligned reads. Variants may include single nucleotide permutations (SNPs; also known as single nucleotide variants, SNVs), insertions or deletions (INDELs), copy number variants (CNVs), as well as large rearrangements, substitutions, duplications, translocations, and others. Preferably variant calling is robust enough to sort out the real variants from the amplification and sequencing noise artefacts.

### Design of Capture Probes

The present disclosure may be directed to a method for detecting variants within one or more genes of interest. In an embodiment, a set of capture probes may be designed to cover at least one gene of interest, which may include the coding regions (exons) and the non-coding regions (introns and UTRs). In some embodiments, capture probes may be at least between 10 base pairs (bp) and 150 bp in length. In some embodiments, capture probes may be at least 10 bp in length. In some embodiments, capture probes may at least be 15 bp in length. In some embodiments, capture probes may be at least 20 bp in length. In some embodiments, capture probes may be at least 25 bp in length. In some embodiments, capture probes may be at least 30 bp in length. In some embodiments, capture probes may be at least 35 bp in length. In some embodiments, capture probes may be at least 40 bp in length. In some embodiments, capture probes may be at least 45 bp in length. In some embodiments, capture probes may be at least 50 bp in length. In some embodiments, capture probes may be at least 55 bp in length. In some embodiments, capture probes may be at least 60 bp in length. In some embodiments, capture probes may be at least 65 bp in length. In some embodiments, capture probes may be at least 70 bp in length. In some embodiments, capture probes may be at least 75 bp in length. In some embodiments, capture probes may be at least 80 bp in length. In some embodiments, capture probes may be at least 85 bp in length. In some embodiments, capture probes may be at least 90 bp in length. In some embodiments, capture probes may be at least 95 bp in length. In some embodiments, capture probes may be at least 100 bp in length. In some embodiments, capture probes may be at least 105 bp in length. In some embodiments, capture probes may be at least 110 bp in length. In some embodiments, capture probes may be at least 115 bp in length. In some embodiments, capture probes may be at least 120 bp in length. In some embodiments, capture probes may be at least 125 bp in length. In some embodiments, capture probes may be at least 130 bp in length. In some embodiments, capture probes may be at least 135 bp in length. In some embodiments, capture probes may be at least 140 bp in length. In some embodiments, capture probes may be at least 145 bp in length. In some embodiments, capture probes may be at least 150 bp in length.

In some embodiments, highly-repetitive sequences may be masked from the gene of interest. Further, the extremities of the highly-repetitive regions may be kept, to allow capture based on adjacent sequences. In some embodiments, the length of highly-repetitive sequences included may be between 10 bp and 20 bp. It will be apparent to those skilled in the art that other lengths are possible. For instance, up to about half of the probe length, although inclusion of longer segments from the repetitive sequences in the probes may increase the risk of off-target captures.

In one embodiment, the capture probes may be designed based on a reference genome. In such an embodiment, capture probes may cover most of the gene sequence, including exons, introns, and untranslated regions (UTRs). In another embodiment, the capture probes may be designed based on a reference genome so that they cover regions presenting a limited number of structural polymorphisms in populations, which may be accomplished through the use of pre-existing databases.

In some embodiments, the capture probes may be designed based on a reference genome so that they cover positions that were evaluated as providing a consistent coverage within and/or among individuals in a previous study. In yet another embodiment, a pangenome reference, which captures structural variants detected among a diversity of individuals, may be used to capture a higher diversity of alternative alleles.

In some embodiments, the probes may represent a combination of different sets of probes. The first set of probes may be placed as evenly as possible across the defined regions of interest so that there is no probe coverage gap and the resulting tiling is as close to 1× as possible.

In another embodiment, at least one other set of probes may be designed by placing evenly and symmetrically an additional one, two, four and/or a different number of probes in between consecutive pairs of probes from the first set. Moreover, probes from any of the sets targeting repetitive regions, which can lead to high rates of off-target reads, may be identified and marked as risky probes.

In some embodiments, risky probes may be identified as those corresponding to regions with a low mappability score, using predefined mappability score values across the genome. In said embodiments, the mappability score may be defined as one divided by the number of places in the genome where a sequence that is nearly identical to a sequence surrounding the position is found.

In some embodiments, the identification of risky probes may require aligning the individual probes to a reference genome with pairwise sequence alignment tools, such as BLAT, and flagging those aligning to a number of genomic regions above a given threshold. Additionally, the risky probes may be removed from the probe set. In other embodiments, the risky probes may be used but kept as a different reagent that is subsequently combined with non-risky probes to monitor their effect.

In some embodiments, the probes that are the riskiest (based on their number of repeats and/or similarity among repeats) may be discarded, while the remaining risky probes are used, but kept as a different reagent that is then monitored with non-risky probes to monitor their effect.

In some embodiments, the designed probes may be synthesized as biotinylated DNA, and may be referred to as baits, to be used during library preparation with hybrid-capture target enrichment. In some embodiments, biotinylated baits may be captured using magnetic beads coated with streptavidin. In some embodiments, the designed probes may be synthesized with other affinity tag as baits. In some embodiments, the other affinity tag capture systems include, but are not limited to, digoxigenin (DIG) capture systems, His-tag capture systems, click chemistry capture systems, antibody-mediated nucleic acid captures systems.

In some embodiments the designed probes may be synthesized such that they are compatible with direct-surface immobilized capture systems, wherein capture probes are immobilized directly to a solid surface. In some embodiments, the probes may be combined, before or after their synthesis, with probes that target other genes and regions of interest.

In some embodiments, the designed capture probes may be utilized to generate a capture library. In some embodiments, the designed capture probes target the PTEN gene. In some embodiments, the designed capture probes target the whole length of the PTEN gene. In some embodiments, the designed capture probes target the coding region of the PTEN gene. In some embodiments, the designed capture probes target the PTEN gene and at least one other gene including, but not limited to, AKT1, ATM, BARD1, BRCA1, BRCA2, BRIP1, CCNE1, CDK12, CHEK1, CHEK2, ESR1, FANCA, FANCD2, FANCL, FGFR1, FGFR2, FGFR3, MRE11, NBN, PALB2, PIK3CA, PPP2R2A, RAD51B, RAD51C, RAD51D, RAD54L, and TP53. In some embodiments, the designed capture probes target the whole length of the PTEN gene and coding regions from 27 other genes; AKT1, ATM, BARD1, BRCA1, BRCA2, BRIP1, CCNE1, CDK12, CHEK1, CHEK2, ESR1, FANCA, FANCD2, FANCL, FGFR1, FGFR2, FGFR3, MRE11, NBN, PALB2, PIK3CA, PPP2R2A, RAD51B, RAD51C, RAD51D, RAD54L, and TP53.

In some embodiments, probes might be desired to capture paralogs of targeted genes (e.g., pseudogenes), capture important non-coding markers or resolve known structural variants. Finally, probes might be added to reduce problems in coverage heterogeneity. The large number of probes typical of large panels may render customization unpracticable, but the present disclosure herein may provide an efficient combination of probes for large panels with customized and/or improved probes within a single assay.

### Data Generation Workflow

The capture probes may be used to prepare a sequencing library using one of the hybrid capture target-enrichment library preparation workflows known to those skilled in the art.

In some embodiments, targeted enrichment may be implemented using amplicon-based enrichment. This method uses primers designed to amplify specific DNA regions of interest through PCR. In some embodiments, targeted enrichment may be implemented using hybridization capture-based enrichment. This method uses probes that are complementary to the target regions. These probes hybridize to the DNA sample, and the target regions are isolated. In some embodiments, isolation techniques include the use of biotinylated probes that bind to streptavidin beads, and/or the use of magnetic beads.

In yet a further embodiment, at least one additional set of probes may be designed to improve capture, for example by targeting alternative alleles that differ strongly from the reference genome, by adding probes to capture pseudogenes, by adding probes to decrease coverage heterogeneity, and the like.

In embodiments where different sets of probes are designed, the different sets may be mixed to obtain a single set of probes. In an additional embodiment, two distinct sets of probes, both corresponding to different sets of genomic regions of special interest, may be designed. It should be apparent to those having ordinary skill in the art that more than two sets of probes can be designed.

Moreover, different coverages may be targeted for each set of probes. For example, a given coverage may be required to identify different variants. Examples of said variants may include single-nucleotide variants (SNVs), insertions and deletions (indels), copy number variations (CNVs), and the like.

A whole-genome sequencing (WGS) library may be prepared from starting DNA or TNA, using methods known to those skilled in the art. DNA isolation, DNA extraction, or DNA purification methods include, but are not limited to any known method to the skilled artisan, e.g., lysing extracted DNA from a sample using e.g., a detergent (e.g., sodium dodecyl sulphate, Triton X-100), separating the soluble DNA from the cell debris and other insoluble material, binding the DNA of interest to a purification matrix (e.g., silica), wash the bound DNA to remove impurities, and elute the bound DNA from the purification matrix. If needed, the isolated DNA is divided into multiple aliquots and each one is individually processed.

The nucleic acids might be extracted from a fresh or a fresh-frozen sample, which might be blood sample, a saliva sample, or a sample from another tissue, for example obtained via biopsy. The nucleic acids might alternatively be isolated from a formalin-fixed paraffin-embedded (FFPE) sample, for example after a tumor biopsy. In other embodiments, the nucleic acids might be cell-free DNA (cfDNA) and/or cell-free RNA (cfRNA) isolated from a bodily fluid, such as blood, blood plasma, urine, or cerebrospinal fluid. The cfDNA/cfRNA might contain circulating tumor DNA (ctDNA), circulating tumor RNA (ctRNA) or fetal DNA.

The DNA or TNA may be extracted from blood samples, from fresh-frozen samples, from formalin-fixed paraffin-embedded (FFPE) samples, or from cell-free DNA (cfDNA) obtained from bodily fluids, such as blood plasma, saliva, or urine.

In some embodiments, the WGS library preparation may start with fragmentation of the DNA, via mechanical or enzymatic fragmentation. Fragmentation may result in a fragmented DNA being 50 to 10000 base-pairs in length. In some embodiments, fragmentation may result in fragmented DNA being 50 base-pairs to 500 base-pairs in length. In some embodiments, fragmentation may result in fragmented DNA being 500 to 1000 base-pairs in length. In some embodiments, fragmentation may result in fragmented DNA being 1000-2000 base-pairs in length. In some embodiments, fragmentation may result in fragmented DNA being 2000-3000 base-pairs in length. In some embodiments, fragmentation may result in fragmented DNA being 3000-4000 base-pairs in length. In some embodiments, fragmentation may result in fragmented DNA being 4000-5000 base-pairs in length. In some embodiments, fragmentation may result in fragmented DNA being 5000-6000 base-pairs in length. In some embodiments, fragmentation may result in fragmented DNA being 6000-7000 base-pairs in length. In some embodiments, fragmentation may result in fragmented DNA being 7000-8000 base-pairs in length. In some embodiments, fragmentation may result in fragmented DNA being 8000-9000 base-pairs in length. In some embodiments, fragmentation may result in fragmented DNA being 9000-10000 base-pairs in length.

Fragmentation can be performed using any method known to the skilled artisan, including, but not limited to, mechanical shearing, sonication, ultrasonication, enzymatic fragmentation, partial digestion, restriction enzyme digestion. The fragmentation step may be skipped if the starting material is already present as small fragments, as is for example typical of such as cDNA, cfDNA, cfRNA, and DNA isolated from some FFPE samples.

The ends of the double-stranded DNA fragments might be repaired and a hanging A′ may be added to facilitate ligation of an adaptor. After fragmentation, the extracted DNA can be end-repaired or end-polished and a single adenine base can be added to form an overhang by an A-tailing reaction. This A-overhang allows adapters containing a single thymine base to pair with the DNA fragments.

Adaptors may then be ligated to both ends of each end-repaired double-stranded DNA fragments. In some embodiments, the adaptors may include exogenous molecular identifiers, with a number of distinct identifiers determined by the needs of the assay. In some embodiments, the molecular identifier tags may be 3 base pairs in length. In some embodiments, the molecular identifier tags may be 4 base pairs in length. In some embodiments, the molecular identifier tags may be 5 base pairs in length. In some embodiments, the molecular identifier tags may be 6 base pairs in length. In some embodiments, the molecular identifier tags may be 7 base pairs in length. In some embodiments, the molecular identifier tags may be 8 base pairs in length. In some embodiments, the molecular identifier tags may be 9 base pairs in length. In some embodiments, the molecular identifier tags may be 10 base pairs in length. In some embodiments, the molecular identifier tags may be 11 base pairs in length. In some embodiments, the molecular identifier tags may be 12 base pairs in length. In some embodiments, the molecular identifier tags may be 13 base pairs in length. In some embodiments, the molecular identifier tags may be 14 base pairs in length. In some embodiments, the molecular identifier tags may be 15 base pairs in length. In some embodiments, the molecular identifier tags may be 16 base pairs in length. In some embodiments, the molecular identifier tags may be 17 base pairs in length. In some embodiments, the molecular identifier tags may be 18 base pairs in length. In some embodiments, the molecular identifier tags may be 19 base pairs in length. In some embodiments, the molecular identifier tags may be 20 base pairs in length.

In some embodiments, the molecular identifier tags and/or the adapters may comprise modified nucleic acids. Non-limiting examples of modified nucleic acids comprise locked nucleic acids (LNAs), which can fine-tune sequence melting temperatures, hybridization stability, resist degradation, etc., peptide nucleic acids (PNAs), which may enhance binding affinity, resist enzyme degradation, etc., 2′-O-methyloxy-ethyl bases (2′-MOE), which may offer increased binding affinity and resist nuclease degradation, fluorobases, which have a fluorine modified ribose for increased binding affinity, 5-hydroxybutynl-2′-deoxyuridine, which is a duplex-stabilizing modified base, and 8-aza-7-deazaguanosine, which is a modified base that eliminates secondary structures associated with GC-rich sequences.

The adaptor-ligated DNA fragments may then by amplified by PCR. The PCR primers might match the adapters on their 3′ ends, but be longer on their 5′ ends, for example to incorporate sample barcodes to the DNA fragments. After clean-up, the resulting collection of adaptor-ligated DNA fragments constitutes a WGS library. In some embodiments, the WGS library may be prepared using a PCR-free protocol.

Part, or the entirety, of the WGS library may be hybridized to the capture probes to prepare a capture library. For example, preparation of the capture library may involve binding to streptavidin beads, extraction of DNA-bead complexes, and/or purification of the complexes. A PCR amplification may be conducted after the capture. Following removal of undesired reagents, the amplified DNA fragments constitute the capture library. It will be apparent to those skilled in the art that variation in the target-enrichment method may be implemented.

In some embodiments, part of the purified WGS library is mixed with the capture library, so that all genomic regions may be represented in the final library, although at a generally lower concentration for regions not targeted by the capture probes. This approach allows the combined generation of a low-pass WGS and target-enriched high-coverage sequence for selected regions.

The prepared library, which may contain some of the WGS library, in addition to the target-enrichment library, is subjected to high-throughput sequencing. To illustrate, the high-throughput sequencing may be accomplished via sequencing-by-synthesis, sequencing-by-ligation, or the like.

In some embodiments, part of the purified WGS library is sequenced in parallel to the capture library. In such embodiments, the WGS and capture libraries may be sequenced as part of distinct sequencing runs, or as part of the same sequencing runs after having received distinct sample barcodes. While this approach increases the handling time and sequence costs, it has the advantage of allowing distinguishing reads originating in the WGS library from those originating in the capture library after sequencing.

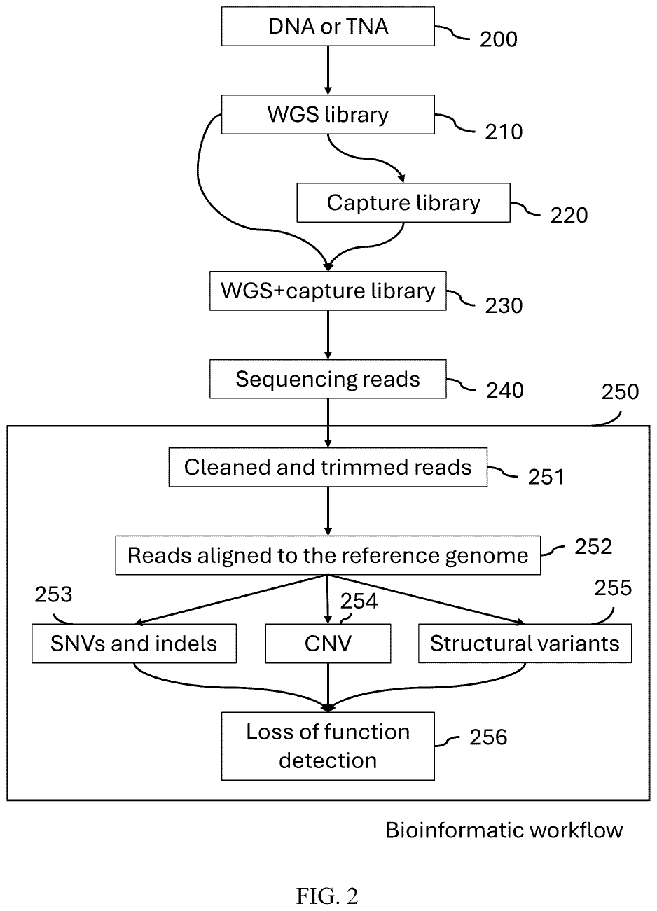

FIG. 2 provides a schematic of a non-limiting embodiment of the workflow. The input material consists in DNA or TNA 200, which may be derived from a patient suffering from, or suspected to suffer from, a disease, such as cancer. The DNA/TNA is processed to prepare a whole-genome sequencing (WGS) library 220. Part of the WGS library may be used to produce a capture library 220. Part of the original WGS library 210 may be mixed with the capture library 220 to produce a WGS+capture library 230. After high-throughput sequencing, sequencing reads 240 are obtained. The sequencing reads can be processed by the bioinformatic workflow 250, which might be provided by a genomic platform. First, the reads are cleaned and trimmed to produce cleaned and trimmed reads 251. Then, the reads may be aligned to the reference genome 252. Different types of variants may be inferred from the aligned reads 252; single nucleotide variants (SNVs) and short indels 253, CNVs 254, and structural variants 255. The different types of variants may finally be combined to detect potential losses of function 256.

### Bioinformatic Workflow

Sequencing reads may subsequently be subjected to filtering and trimming, to remove low-quality reads, low-quality parts of reads, adapters, and the like. The cleaned sequencing reads may be aligned to a reference genome, using methods known to those skilled in the art. In embodiments where the capture library and WGS library were pooled before sequencing, the reads corresponding to genomic regions targeted by the capture probes may be separate and considered as capture reads, even though they will contain some WGS reads. The reads mapping to regions not targeted by the capture probes may conversely be considered as WGS reads, although they might contain some off-target capture reads. In another embodiments, the reference genome may be divided into bins of pre-set or variable size, and reads may be considered as WGS reads if they map to a genomic bin not overlapping with any region targeted by the capture probes. As a non-limiting example, the genome may be divided into non-overlapping bins of 1,000, 10,000, 100,000 or 1,000,000 bp.

In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp). In some embodiments, the sequence reads are of a mean, median, or average length of about 1000 bp, 2000 bp, 5000 bp, 10,000 bp, or 50,000 bp or more.

Nanopore® sequencing methods and associated devices provided by Oxford Nanopore Technology PLC of Oxford, UK, for example, can provide sequence reads that can vary in size from tens to hundreds to thousands of base pairs. Illumina® parallel sequencing methods and associated devices provided by Illumina Inc. of San Diego, CA, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

The cleaned and aligned reads may be assigned to read families representing potential PCR duplicates. In the absence of exogenous molecular identifiers, the assignment may be based on endogenous molecular identifiers, such as the start and end positions of the reads with respect to the reference genome and/or the sequence of the reads. In the presence of exogenous molecular identifiers, the molecular identifiers may be identified and trimmed during the read cleaning step. The exogenous molecular identifiers present at each end of each pair of sequencing reads may be converted into numerical codes, and pairs of reads sharing the same numerical codes may be assigned to the same read family. In some embodiments, the assignment of reads to read families encompassing potential PCR duplicates may consider both endogenous and exogenous molecular identifiers.

As an example, pairs of reads may be assigned to the same read family if they share the same numerical code and the same start and end positions with respect to the reference genome. The exogenous molecular identifiers, potentially combined with endogenous barcodes, may moreover be used to recognize reads originating from each of the two strands in the original molecules (′plus' and ‘minus’ strands, also known as ‘Watson’ and ‘Crick’ strands). Indeed, reads originating from each of the two strands in the same original molecule will share the same exogenous barcodes, but on opposite ends.

The cleaned reads aligned to the reference genome may be used to call variants, such as single-nucleotide variants (SNVs), short insertions and deletions (indels), copy-number variants [e.g. Ivanov et al.; Patent Pub. No. US 2022/0310488], or genomic metrics, including a genomic instability index [e.g. Pozzorini et al. Patent Pub. No. WO 2022/023381], tumor mutational burden [e.g. Bieler et al. Patent Pub. No. EP 4207204], microsatellite instability [e.g. Song et al. Patent Pub. No. WO 2021/156486], and the like.

In embodiments where reads have been assigned to read families, variant calling may be performed using a probabilistic variant calling approach or a consensus sequencing approach. In embodiments where both capture and WGS libraries were sequenced, whether separately or after pooling, different markers may be evaluated with one or the other subset of reads. As a non-limiting example, SNVs, indels and microsatellite instability may be evaluated based on capture reads, while a genomic instability index may be calculated based on WGS reads. As another non-limiting example, some markers (e.g., SNVs and indels) may be assessed based solely on capture reads, while others (e.g., copy-number variants) may be assessed based on both capture and WGS reads.

In the context of the present disclosure, analytical methods specifically developed to detect intra-genic structural variants and assessed holistic loss of function are disclosed.

The methods disclosed herein may include methods to infer the presence of insertions and deletions and other structural variants, which increases the accuracy in genes that contain few exons, short exons, a high fraction of intronic sequences, and the like. It will be apparent to those skilled in the art that the novel analytical methods described herein may be applied to the detection of structural variants from data generated with WGS or long-read target enrichment with probes in the exonic sequences, in addition to short-read sequence data generated from a target-enrichment library obtained with probes designed with the method disclosed above.

In some embodiments, coverage may be computed from the cleaned reads aligned to the at least one gene of interest and sites or genomic regions providing a uniform and/or stable coverage among replicates. Alternatively, individual sites or regions may be selected for subsequent analyses. In other embodiments, all sites with coverage above a given threshold may be retained for analysis. Other embodiments may involve other filtering and quality checks.

Copy-number variants may be detected at the gene level, or even genomic region level, by considering the coverage of the gene or genomic regions, based on the number of cleaned mapped reads and the length of the gene or genomic region. The coverage may be normalized across genes and/or genomic regions, for instance by dividing the coverage of each region by the mean or median coverage across all regions. The coverage may further by normalized across samples, for example by dividing the coverage of each region by the mean or median coverage observed among other samples for the same region. The normalization may further include corrections for GC content, mappability, or other features susceptible of altering the observed coverages. Copy numbers may finally be inferred from the normalized coverage data. In some embodiments, inferring copy numbers may be done using a hidden-Markov model (HMM). In some embodiments, the inferring of copy numbers may be performed alongside the coverage normalization, for example using an iterative process that performs each new normalization considering previously inferred copy number [e.g. Ivanov et al.; Patent Pub. No. US 2022/0310488].

Additionally, copy-number variants may be detected at the exon level. In an embodiment, the coverage inferred from sequence data per exon may be converted to remove sample-specific and/or region-specific coverage biases, using cross-sample normalization, cross-region normalization, correction for GC content, and/or other methods known to those skilled in the art. In the present disclosure, virtual exons may be defined within the intronic regions. The number of virtual introns may exceed the number of real exons, which may occur, for example, in the case of large introns allowing multiple virtual exons. It will be apparent to those skilled in the art that various criteria may be used to establish the number of virtual exons and their limits, such as the sequence conservatism of the region, the coverage uniformity of the region, the distance to other defined regions including exons, and the like.

The coverage across successive exons, including both genuine and virtual exons, may subsequently be employed to infer changes in coverage. To illustrate, hidden Markov models previously applied to copy number variant inference in an iterative scheme [Ivanov et al.; US Patent Pub. No. US 2022/0310488] may be used to infer said changes. The presence of virtual exons may increase the total number of exons in the model and therefore the ability to detect changes in coverage, especially when the number of genuine exons is limited. The results may subsequently be reported either solely for genuine exons or for both genuine exons and virtual exons. In an embodiment, the latter may represent intronic segments.

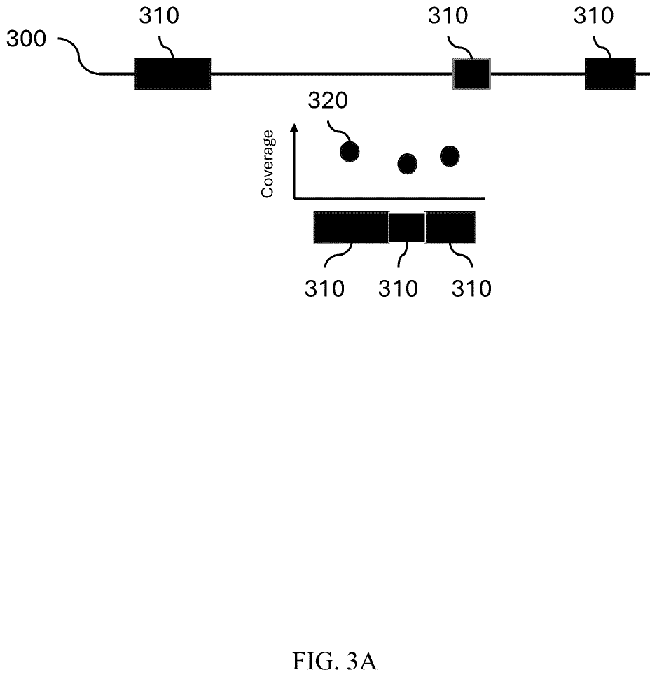

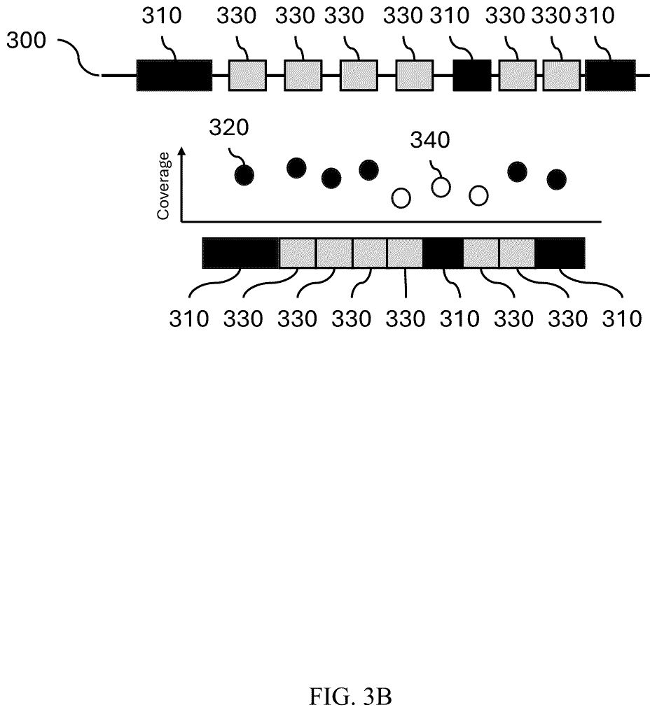

FIG. 3 illustrates the effect of virtual exons on exon-level inference. In FIG. 3A, only real exons are used. A gene 300 is illustrated, with three exons 310 separated by non-coding DNA. Their coverage 320 is plotted, and while the second exon has a lower coverage, it might not be significantly lower than variation expected by chance in the presence of experimental noise. In FIG. 3B, the same analysis is performed in the presence of virtual exons 330 in addition to real exons 310. The coverage plot might now suggest a stretch of lower coverages represented by white points 340 among higher coverages represented by black points 320. The algorithm might therefore be able to detect a deletion stretching the second exon and parts of the introns.

In some embodiments, the detection of CNVs may be based on WGS, which may be low-pass WGS data. As non-limiting examples, the low-pass WGS data may have coverages below 30×, below 5×, or below 1×. In some embodiments, the detection of CNVs may be based on target-enrichment sequence data. It will be apparent to those trained in the art that the lower coverage of low-pass WGS may prevent the detection of fine-scale CNVs, such as exon-level detection. Analyses based on the two types of data may however be combined to obtain confirmations of results. As a non-limiting example, CNVs inferred with low confidence from target-enrichment data may be confirmed with low-pass WGS. In some cases, low-pass WGS might detect CNVs that may be below the threshold of confidence from target-enrichment data. In addition, low-pass WGS data may help distinguish homozygous from heterozygous deletions. Indeed, coverage analyses from target-enrichment data are subject to strong biases, reflecting the additional steps in the data generation workflow. By contrast, low-pass WGS data present less coverage biases. In cases where target-enrichment data do not allow distinguishing between loss of one copy or loss of two copies, the addition of low-pass WGS may therefore help differentiate the two scenarios.

In some embodiments, the low-pass WGS data may be used specifically to detect large-scale copy-number variants that may stretch thousands of base pairs up to entire chromosomes. The coverage may be calculated per genomic bin, as previously defined, by computing the number of reads mapped to each bin. The coverage may then be normalized by dividing the coverage of each bin by the median or mean coverage among bins. Normalization may moreover control for GC-content variation among bins, for example by taking the residuals of a regression between the bin coverages and the bin GC contents, or other methods known in the art. Variation in copy number among bins can then be detected from the normalized coverages using various methods, including hidden-Markov models used previously [e.g. Ivanov et al.; Patent Pub. No. US 2022/0310488]. The models can moreover be adapted to consider the tumor purity of the sample, or other factors. It is also possible to use machine-learning models, such as convolutional neural networks, to classify coverage patterns after training the model based on a dataset with known copy numbers. Other methods known in the art may be used to detect copy number variants from low-pass WGS coverage profiles.

In some embodiments, the low-pass WGS data may be analyzed independently to infer genetic variants that might be missed by the target-enrichment data. Indeed, low-pass WGS might detect variants corresponding to alleles that are not captured by the probes due to sequence divergence preventing binding. In other cases, low-pass WGS might detect variants corresponding to alleles outside of the probe design.

In some embodiments, the exon-level copy number estimates may be combined with gene-level copy number estimates to infer copy number variation within genes in addition to copy number variation of the whole gene. As a non-limited example, copy number variant (CNV) detection may be performed independently at the gene level and the exon level. The gene level detection may be performed with the total coverage for each gene, potentially normalized among genes within each sample (by dividing the coverage of each gene by the mean or median coverage for the gene) and/or among samples for each gene (by dividing the coverage of each gene by the median or mean coverage of the gene among samples).

The normalization may be done in an iterative way, while optimizing copy numbers within each iteration [Ivanov et al.; Patent Pub. No. US 2022/0310488]. The result of the gene-level CNV analysis may be used to classify the gene into copy gain, copy loss, or no copy change, with the potential addition of potential copy gain and potential copy loss. The classification may be based on thresholds, either predefined or optimized based on the dataset. The exon-level status may be determined using the same methods, but with the coverage calculated, and potentially normalized, by exon, which may include virtual exons delimited within introns. Different decision trees or algorithms may then be used to combine the gene-level and exon-level analyses. In some embodiments, the gene-level status may be assigned to the gene if no exon-level variation is detected. If exon-level CNVs are detected, then decision trees may be used. Exons may first be grouped into groups with the same assigned coverage level. As a non-limiting example, a gene-level status may be then assigned using the following rules: (i) if the gene-level status is ‘copy gain’ and the minimum coverage among the groups of exons is above the threshold for copy gain, the gene level status removes as ‘copy gain’; (ii) if the gene-level status is ‘copy loss’ and the maximum coverage among the groups of exons is below the threshold for copy loss, then the gene status remains as ‘copy loss; and (iii) in other cases, an exon-level event is suspected.

For the exon-level status, the number of groups of consecutive exons assigned to distinct coverage may first be considered. If only two groups are defined, the following rules may apply: (i) if the coverage of one group is significantly smaller than 2 while the coverage of the other group is not significantly different from 2, then an exon loss is inferred; (ii) if the coverage of one group is significantly greater than 2 while the coverage of the other group is not significantly different from two, then an exon gain is inferred; and (iii) in other cases, the event may remain uncharacterized, although detected.

It will be apparent to those trained in the art that different methods can be used to assess the significance of the deviation from a coverage of 2, such as a pre-defined threshold, a threshold calculated based on a reference dataset, or a p-value for the significance of the deviation, using a statistical or an empirical expected distribution.

In cases where more than two groups with distinct coverages are identified, other rules may be used. As a non-limiting example, if three groups are identified and the group positioned in the middle of the gene has a higher coverage, an exon gain may be inferred. As another non-limiting example, if three groups are identified and the group positioned in the middle of the gene has a lower coverage, an exon loss may be inferred. Other rules may be used to capture a higher number of potential cases.

In all embodiments, the output of the gene-level and exon-level CNV analysis is the identification of gene-level copy number variants complemented by an identification of intra-genic copy number variants.

Further, structural variants may be identified based on the presence of discordant gene pairs or reads spanning sequences that are not contiguous within the reference genome. For instance, algorithms may be deployed to detect discordant gene pairs or reads spanning non-contiguous reference sequences in numbers that allow confident inference. It will be apparent to those skilled in the art that the confidence level may be established based on: (1) pre-determined thresholds; (2) thresholds adjusted from the data; and/or (3) statistical significance compared to a null model expectation. Structural variants may subsequently be inferred when the observed discordant gene pairs and/or reads spanning non-contiguous reference sequences exceed the threshold.

Some individual genetic variants may be sufficient to create a heterozygous loss of function. This includes gene losses, but also partial gene losses, gene rearrangement, such as intra-genic duplications, inversions, translocations, or gene fusions. Single nucleotide variants (SNVs) and short insertion/deletions (indels) can also create loss of function if they disrupt the start codon or insert premature STOP codons, as will among others be the case of indels shifting the reading frame of the gene. In addition, non-synonymous SNVs changing the encoded amino acid can significantly alter the protein function or stability even without inserting STOP codons. Prediction of the effect of a SNV on the protein can therefore be used to detect SNVs likely causing a loss of function or drastic protein changes equivalent to a loss of function. In this context, the prediction of pathogenicity, using one of the methods known in the art, may be considered as indicating a SNV is likely causing a loss of function.

In some embodiments, the information provided by different analytical tools may be combined to infer biological processes. As a nonlimiting example, estimates of gene-level copy number variants and exon-level copy number variants may be combined with read-based estimates of structural variants to infer rearrangements. Specifically, rearrangements may be inferred by reporting high-confidence structural variants when they are detected with both methods. As a further nonlimiting example, the presence of alterations in a gene of interest causing a loss of function may be inferred if at least one of a gene-level copy loss, an exon-level copy number, a structural variant representing a large deletion, a tandem duplication of a large part of the gene or a fusion with a different gene, and/or a single nucleotide variant altering the encoded protein is detected.

As a non-limiting example, a loss-of-function might be inferred for a gene if any of the following is detected: (i) partial or complete gene deletion; (ii) tandem duplication, inversion, translocation, or gene fusion; and (iii) frameshift mutations, stop codons or pathogenic amino acid substitutions.

Homozygous loss of function may be inferred when mutations causing loss of function can be assigned to distinct alleles. As a non-limiting example, homozygous loss of function may be inferred if coverage data is compatible with the complete absence of a gene, or part of a gene, after accounting for the fact that the sample may contain tumor tissue in addition to non-tumor tissue. As a further non-limiting example, a homozygous loss of function may be inferred if SNVs or indels causing loss of function can be assigned to distinct alleles after read phasing. As a further non-limiting example, a homozygous loss of function may be inferred if coverage data support the loss of a gene or part of a gene and other loss-of-function causing variants are detected in reads overlapping the lost gene or part of gene and must therefore be attributed to the other allele. Those trained in the art will understand that other patterns can support assigned loss-of-function causing variants to distinct alleles. It will also be apparent that a complete loss of function will not be inferred in a sample with more than two gene copies, where not all of them harbor loss-of-function causing variants.

Whether the loss-of-function concerns one or two of the alleles may be inferred by combining information from the low-pass WGS data, the target-enrichment data, and phasing analyses. In the context of gene losses, variants, including copy-number variants, detected based on low-pass WGS can be incorporated with variants detected based on target enrichment to detect loss of function of specific genes, or differentiate heterozygous and homozygous deletions. As a non-limiting example, genes in which intra-specific deletions are detected based on target enrichment data can be classified as having a homozygous loss of function if low-pass WGS indicates a large-scale deletion on the other allele.

### Computer Systems to Implement the Methods Described Herein

The implementation of the bioinformatic workflow disclosed here requires a genomic platform, which may consist of one or multiple computer processes running one or multiple communication computer programs. In some embodiments, a single genomic platform may be used to conduct all the steps, while in other embodiments, different genomic platforms may be responsible for distinct parts of the workflow. In some embodiments, the genomic platform may moreover provide a user interface, allowing the user to access and explore the full breadth of results. In some embodiments, the user interface may moreover allow the user to produce automatic or customized reports, and to export, download, or transfer to another system such reports. One example of such a genomic platform is SOPHIA DDM™, produced by SOPHIA GENETICS. The methods disclosed here are however compatible with other genomic platforms, including custom-made ones.

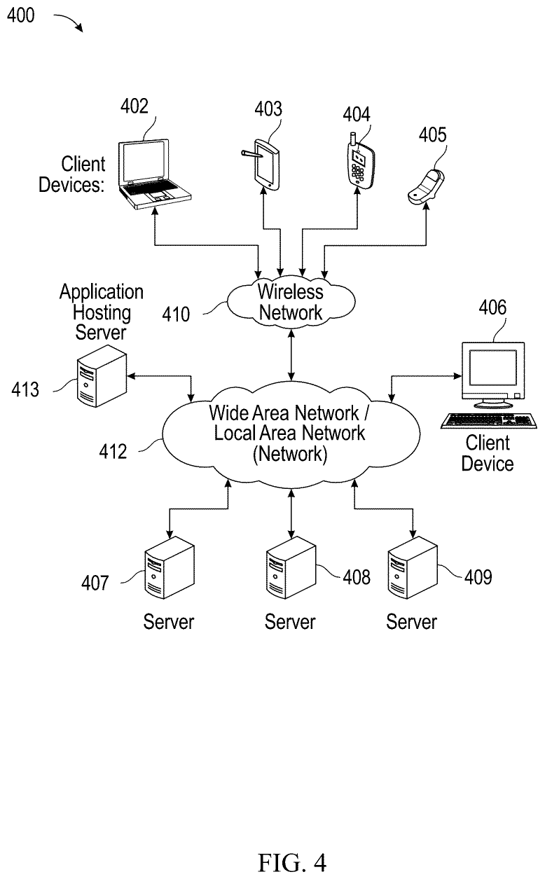

FIG. 4 illustrates components of one embodiment of an environment in which the dry-lab steps of the present disclosure may be practiced. Not all of the components may be required to practice the present disclosure, and variations in the arrangement and type of the components may be made without departing from the spirit or scope of the present disclosure.

As shown, the system 400 includes one or more Local Area Networks (“LANs”)/Wide Area Networks (“WANs”) 412, one or more wireless networks 410, one or more wired or wireless client devices 406, mobile or other wireless client devices 402-405, servers 407-409, and may include or communicate with one or more data stores or databases. The client devices 402-406 may include, for example, at least one of desktop computers, laptop computers, set top boxes, tablets, cell phones, smart phones, smart speakers, wearable devices (such as the Apple Watch) and the like. Servers 407-409 can include, for example, one or more application servers, content servers, search servers, and the like. FIG. 4 also illustrates application hosting server 413.

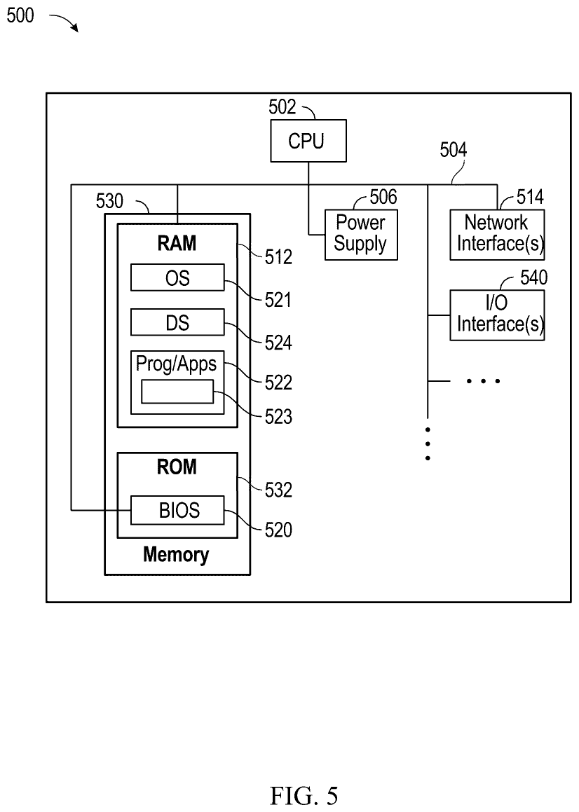

FIG. 5 illustrates a block diagram of an electronic device 500 that can implement one or more aspects of an apparatus, system, and method for measurement and secure transmission of physical properties (the “Engine”) according to one embodiment of the present disclosure. Instances of the electronic device 500 may include servers, e.g., servers 407-409, and client devices, e.g., client devices 402-406. In general, the electronic device 500 can include a processor/CPU 502, memory 530, a power supply 506, and input/output (I/O) components/devices 540, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 500. A touchscreen may determine whether a user is providing input by, for example, determining whether the user is touching the touchscreen with a part of the user's body such as his or her fingers. The electronic device 500 can also include a communications bus 504 that connects the aforementioned elements of the electronic device 500. Network interfaces 514 can include a receiver and a transmitter (or transceiver), and one or more antennas for wireless communications.

The processor 502 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 530, which can include Random Access Memory (RAM) 512 and Read Only Memory (ROM) 532, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 521, data storage 524, which may include one or more databases, and programs and/or applications 522, which can include, for example, software aspects of the program 523. The ROM 532 can also include Basic Input/Output System (BIOS) 520 of the electronic device.

Software aspects of the program 523 are intended to broadly include or represent all programming, applications, algorithms, models, software, and other tools necessary to implement or facilitate methods and systems according to embodiments of the present disclosure. The elements may exist on a single computer or be distributed among multiple computers, servers, devices, or entities.

The power supply 506 contains one or more power components and facilitates supply and management of power to the electronic device 500.

The input/output components, including Input/Output (I/O) interfaces 540, can include, for example, any interfaces for facilitating communication between any components of the electronic device 500, components of external devices (e.g., components of other devices of the network or system 400), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 540 and the bus 504 can facilitate communication between components of the electronic device 500, and in an example can ease processing performed by the processor 502.

Where the electronic device 500 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system, and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 402-406, as may be used in an example apparatus, system and method embodying the Engine, may range widely in terms of capabilities and features. For example, a cell phone, smart phone, or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 402-406, for example, as may be used in an example apparatus, system and method implementing the Engine, may run a variety of operating systems, including personal computer operating systems such as Windows, iOS or Linux, and mobile operating systems such as iOS, Android, Windows Mobile, and the like.

Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 410 or 412, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 410, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatuses, systems, and methods implementing the Engine. Embodiments of the present disclosure may be implemented on one or more of client devices 402-406, which are communicatively coupled to servers including servers 407-409. Moreover, client devices 402-406 may be communicatively (wirelessly or wired) coupled to one another. In particular, software aspects of the Engine may be implemented in the program 523. The program 523 may be implemented on one or more client devices 402-406, one or more servers 407-409, and 413, or a combination of one or more client devices 402-406, and one or more servers 407-409 and 413.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

In some embodiments, the steps of the bioinformatic workflow may be carried out by a software device located on a cloud computing server to permit decentralized analysis. In an embodiment, the cloud computing server may comprise a global center that provides central services, such as user authentication and authorization. In one embodiment, the cloud computing server may comprise at least one regional center to provide file management, storage, and other functionalities. It is contemplated that this permits the users to access the software from a server that complies with local requirements and regulations.

### Kit to Generate the Data Described Herein

Aspects of the present disclosure also include kits. The kits may include, e.g., one or more of any of the components necessary to perform the methods described herein. For example, the kits may include one or more of: at least one set of gene specific primers, at least one adaptor molecule, a polymerase (e.g., a thermostable polymerase, a reverse transcriptase, or the like), ligase (e.g. DNA ligase), dNTPs, a salt, a metal cofactor, NAD, ATP, one or more nuclease inhibitors (e.g., an RNase inhibitor and/or a DNase inhibitor), one or more molecular crowding agents (e.g., polyethylene glycol, or the like), one or more enzyme-stabilizing components (e.g., DTT), or any other desired kit component(s), such as solid supports, e.g., tubes, beads, etc.

The kit may include the reagents needed to produce a WGS library. The kit may include the reagents needed for the fragmentation, such as a fragmentation enzyme and a fragmentation buffer. Nonlimiting examples of fragmentation enzymes include non-specific endonucleases (e.g., DNase I, endonuclease V, MNase, S1 nuclease, fragmentase), transposases (e.g., Tn5 transposase, MuA transposase, Tn7 transposase), restriction endonucleases (e.g., Msel, MspI, TaqI, Alul, HaeIII, EcoRI, PstI, HindIII, BamHI, NotI, Sbfl).

The kit may moreover include reagents to ligate adapters, including Y-shaped adapters, which may contain exogenous barcodes, a ligation enzyme, and a ligation buffer. Nonlimiting examples of a ligation enzyme include T4 DNA ligase, T7 DNA ligase, Taq DNA ligase, E. coli DNA ligase, HiFi Taq DNA ligase.

The kit may additionally include reagents for the post-ligation PCR, such as a polymerase, dNTPs, a buffer, and primers with sample indexes, as well as wash buffer for the post-PCR cleanup. Nonlimiting examples of a polymerase include Taq polymerase, Pfu DNA polymerase, Vent DNA polymerase, Phusion DNA polymerase, Q5 DNA polymerase, Hot-Start DNA polymerases.

The kit may include the reagents to perform the hybrid capture of the WGS library, such as capture probes, universal blockers, human cot DNA, hybridization buffer, hybridization buffer enhancer, streptavidin beads, and bead wash buffer. The kit may moreover include reagents for the post-capture PCR, such as a polymerase, primers, dNTPS, and a PCR buffer, as well as reagents for the post-PCR cleanup, such as a wash buffer.

Components of the kit may be present in separate containers, or multiple components may be present in a single container. For example, the adaptor molecules could be provided pre-aliquoted in separate wells/tubes or attached/encapsulated with different beads, and mixture of all beads is provided as kit components. In certain embodiments, it may be convenient to provide the components in a lyophilized form, so that they are ready to use and can be stored conveniently at room temperature.

In addition to the above-mentioned components, a kit may further include instructions for using the components of the kit, e.g., to practice the methods described herein. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Practical Applications

In some embodiments, the methods described herein may be directed to detecting variants within one or more genes of interest, wherein the one or more genes of interest comprise genes associated with any disease or condition.

In some embodiments, the one or more genes of interest comprise genes associated with cancer. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system cancers (meaning cancers that begin in the tissues of the brain and spinal cord).

Examples of carcinomas include, without limitation, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, an adenocarcinoma, a gastrinoma, a cholangiocarcinoma, a hepatocellular carcinoma, a combined hepatocellular carcinoma and cholangiocarcinoma, a trabecular adenocarcinoma, an adenoid cystic carcinoma, an adenocarcinoma in adenomatous polyp, an adenocarcinoma, familial polyposis coli, a solid carcinoma, a carcinoid tumor, a branchiolo-alveolar adenocarcinoma, a papillary adenocarcinoma, a chromophobe carcinoma, an acidophil carcinoma, an oxyphilic adenocarcinoma, a basophil carcinoma, a clear cell adenocarcinoma, a granular cell carcinoma, a follicular adenocarcinoma, a non-encapsulating sclerosing carcinoma, adrenal cortical carcinoma, an endometroid carcinoma, a skin appendage carcinoma, an apocrine adenocarcinoma, a sebaceous adenocarcinoma, a ceruminous adenocarcinoma, a mucoepidermoid carcinoma, a cystadenocarcinoma, a papillary cystadenocarcinoma, a papillary serous cystadenocarcinoma, a mucinous cystadenocarcinoma, a mucinous adenocarcinoma, a signet ring cell carcinoma, an infiltrating duct carcinoma, a medullary carcinoma, a lobular carcinoma, an inflammatory carcinoma, Paget's disease, a mammary acinar cell carcinoma, an adenosquamous carcinoma, an adenocarcinoma w/squamous metaplasia, a sertoli cell carcinoma, embryonal carcinoma, choriocarcinoma.

Examples of sarcomas include, without limitation, glomangiosarcoma, sarcoma, fibrosarcoma, myxosarcoma, liposarcoma, leiomyosarcoma, rhabdomyosarcoma, embryonal rhabdomyosarcoma, alveolar rhabdomyosarcoma, stromal sarcoma, carcinosarcoma, synovial sarcoma, hemangiosarcoma, kaposi's sarcoma, lymphangiosarcoma, osteosarcoma, juxtacortical osteosarcoma, chondrosarcoma, mesenchymal chondrosarcoma, giant cell tumor of bone, ewing's sarcoma, odontogenic tumor, malignant, ameloblastic odontosarcoma, ameloblastoma, malignant, ameloblastic fibrosarcoma, myeloid sarcoma, mast cell sarcoma.

Examples of leukemias include, without limitation, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, and hairy cell leukemia.

Examples of lymphomas and myelomas include, without limitation, malignant lymphoma, Hodgkin's disease, Hodgkin's, paragranuloma, malignant lymphoma, small lymphocytic, malignant lymphoma, large cell, diffuse, malignant lymphoma, follicular, mycosis fungoides, other specified non-Hodgkin lymphomas, myeloma, and multiple myeloma.

Examples of melanomas include, without limitation, malignant melanoma, amelanotic melanoma, superficial spreading melanoma, malignant melanoma in giant pigmented nevus, and epithelioid cell melanoma.

Examples of brain/spinal cord cancers include, without limitation, pinealoma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, malignant, neurofibrosarcoma, neurilemmoma, malignant.

Examples of other cancers include, without limitation, a thymoma, an ovarian stromal tumor, a thecoma, a granulosa cell tumor, an androblastoma, a leydig cell tumor, a lipid cell tumor, a paraganglioma, an extra-mammary paraganglioma, a pheochromocytoma, blue nevus, malignant, fibrous histiocytoma, malignant, mixed tumor, malignant, mullerian mixed tumor, nephroblastoma, hepatoblastoma, mesenchymoma, malignant, brenner tumor, malignant, phyllodes tumor, malignant, mesothelioma, malignant, dysgerminoma, teratoma, malignant, struma ovarii, malignant, mesonephroma, malignant, hemangioendothelioma, malignant, hemangiopericytoma, malignant, chondroblastoma, malignant, granular cell tumor, malignant, malignant histiocytosis, immunoproliferative small intestinal disease.

In some embodiments, the one or more genes of interest comprise genes associated with brain cancer, spinal cord tumors, breast cancer, lung cancer, tracheal cancer, laryngeal cancer, oropharyngeal cancer, oral cavity cancer, nasopharyngeal cancer, salivary gland cancer, sinus cancer, esophageal cancer, stomach cancer, small intestine cancer, colorectal cancer, anal cancer, liver cancer, intrahepatic bile duct cancer, gallbladder cancer, extrahepatic bile duct cancer, pancreatic cancer, leukemia, lymphoma, multiple myeloma, myelodysplastic syndrome, myeloproliferative neoplasm, bone cancer, soft tissue sarcoma, rhabdomyosarcoma, liposarcoma, osteosarcoma, Ewing sarcoma, melanoma, basal cell carcinoma, squamous cell carcinoma, Merkel cell carcinoma, ovarian cancer, cervical cancer, endometrial cancer, vaginal cancer, vulvar cancer, fallopian tube cancer, prostate cancer, testicular cancer, penile cancer, bladder cancer, kidney cancer, ureter cancer, urethral cancer, thyroid cancer, parathyroid cancer, adrenal cancer, pituitary tumor, neuroendocrine tumor, retinoblastoma, ocular melanoma, neuroblastoma, Wilms tumor, medulloblastoma, hepatoblastoma, or a combination thereof.

Nonlimiting examples of brain cancer include glioblastoma, astrocytoma, oligodendroglioma, ependymoma, medulloblastoma, meningioma, and primary CNS lymphoma. Nonlimiting examples of spinal cord tumors include, intramedullary astrocytoma, ependymoma, and hemangioblastoma.

Nonlimiting examples of breast cancer include invasive ductal carcinoma, invasive lobular carcinoma, ductal carcinoma, lobular carcinoma, triple-negative breast cancer, HER2-positive breast cancer, inflammatory breast cancer, Paget disease of the breast, and metaplastic breast carcinoma. Nonlimiting examples of lung cancer include non-small cell lung cancer (e.g., adenocarcinoma, squamous cell carcinoma, large cell carcinoma), small cell lung cancer, carcinoid tumor, and mesothelioma.

Nonlimiting examples of pancreatic cancer include pancreatic ductal adenocarcinoma, acinar cell carcinoma, pancreatic neuroendocrine tumor, and intraductal papillary mucinous neoplasm. Nonlimiting examples of bone cancer include osteosarcoma, chondrosarcoma, Ewing sarcoma, and giant cell tumor. Nonlimiting examples of soft tissue cancers include liposarcoma, leiomyosarcoma, rhabdomyosarcoma, synovial sarcoma, angiosarcoma, and undifferentiated pleomorphic sarcoma.

Nonlimiting examples of ovarian cancers include high-grade serous carcinoma, low-grade serous carcinoma, endometrioid carcinoma, clear cell carcinoma, mucinous carcinoma, and germ cell tumors. Nonlimiting examples of uterine cancer include endometrioid adenocarcinoma, serous carcinoma, clear cell carcinoma, and uterine carcinosarcoma. Nonlimiting examples of cervical cancers include squamous cell carcinoma, adenocarcinoma, and small cell carcinoma.

Nonlimiting examples of prostate cancers include acinar adenocarcinoma, ductal adenocarcinoma, and small cell carcinoma. Nonlimiting examples of testicular cancer include seminoma, and non-seminomatous germ cell tumors. Nonlimiting examples of thyroid cancer include papillary thyroid carcinoma, follicular thyroid carcinoma, medullary thyroid carcinoma, and anaplastic thyroid carcinoma.

In some embodiments, the methods described herein may facilitate the detection of somatic and/or germline variants within one or more genes of interest associated with therapeutic response, resistance, and prognosis of cancer. In some embodiments, the detected variants within the one or more genes of interest may be used to predict responsiveness to particular cancer treatment. In some embodiments, the cancer treatment comprises a chemotherapy, a radiation therapy, an immunotherapy, a hormonal therapy, a targeted therapy, or a cell therapy.

Nonlimiting examples of chemotherapies include alkylating agents (e.g., altretamine, bendamustine, busulfan, carboplatin, chlorambucil, cisplatin, cyclophosphamide, dacarbazine, ifosfaminde, mechlorethamine, melphalan, oxaliplatin, procarbazine, temozolomide, thiotepa, trabectedin, carmustine, lomustine, streptozocin), antimetabolites (e.g., 5-fluorouracil, 6-mercaptopurine, azacitidine, capaecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine), topoisomerase inhibitors (e.g., etoposide, irinotecan, irinotecan liposomal, mitoxantrone, teniposide, topotecan), mitotic inhibitors (e.g., cabazitaxel, docetaxel, nab-paclitaxel, paclitaxel, vinblastine, vincristine, vincristine liposomal, vinorelbine), antitumor antibiotics (e.g., daunorubicin, doxorubicin, doxorubicin liposomal, epirubicin, idarubicin, mutoxantrone, valrubicin, bleomycin, dactinomycin, mitomycin-c), and other chemotherapies (e.g., all-trans-retinoic acid, arsenic trioxide, asparaginase, eribulin, ixabepilone, mitotane, omacetaxine, pegaspargase, procarbazine, romidepsin, vorinostat, paclitaxel).

Nonlimiting examples of radiation therapy include external beam radiation therapy (e.g., CD conformal radiation therapy, intensity-modulated radiation therapy, image-guided radiation therapy, stereotactic radiotherapy, proton therapy, stereotactic body radiation therapy, volumetric modulated arc therapy, tomotherapy), superficial radiotherapy, intraoperative radiotherapy, and internal radiation therapy (e.g., brachytherapy, interstitial brachytherapy, intracavitary brachytherapy, intraluminal brachytherapy, systemic radiation therapy, radioimmunotherapy, radiopharmaceutical therapies).

Nonlimiting examples of immunotherapy include checkpoint inhibitors (e.g., nivoluman, pembrolizumab, ipilimumab), adoptive cell therapy (e.g., CAR T cell therapy, TIL therapy), monoclonal antibodies (e.g., rituximab, trastuzumab, cetuximab), vaccines (e.g., HPV vaccine, melanoma vaccine), cytokines (e.g., interferon, interleukin), oncolytic viruses (e.g., talimogene la-KS), and immune modulators (e.g., lenalidomide).

Nonlimiting examples of hormonal therapy include aromatase inhibitors (e.g., anastrozole, exemestrane, letrozole), selective estrogen receptor modulators (e.g., tamoxifen), luteinizing hormone-releasing hormone agonists (e.g., goserelin, leuprolide), anti-androgens (e.g., bicalutamide, flutamide, nilutamide), progestins (e.g., medroxyprogesterone, megestrol), androgen synthesis inhibitors (e.g., abiraterone, ketoconazole), and CDK 4/6 inhibitors (e.g., ribociclib, abemaciclin).

Nonlimiting examples of targeted therapy include monoclonal antibodies (e.g., trastuzumab, cetuximab, bevacizumab, pembrolizumab), small molecule drugs (e.g., tyrosine kinase inhibitors, proteasome inhibitors, PARP inhibitors, PI3K inhibitors, AKT inhibitors, mTOR inhibitors), antibody-drug conjugates (e.g., trastuzumab emtansine), and angiogenesis inhibitors.

In some embodiments, the detected variants within the one or more genes of interest may be used to determine whether a subject is a candidate for a CDK 4/6 inhibitor or its biosimilars, including, but not limited to Palbociclib®), ribociclib (Kisqali®), abemaciclib (Verzenio®)

In some embodiments, the detected variants within the one or more genes of interest may be used to determine whether a subject is a candidate for a PI3K inhibitor or its biosimilars, including, but not limited to, alpelisib (Piqray®), idelalisib (Zydelig®), duvelisib (Copiktra®), copanlisib (Aliqopa®), umbralisib (Ukoniq®), leniolisib (Joenja®), inavolisib (Itovebi®), buparlisib, paxalisib, dactolisib, voxtalisib, gedatolisib.

In some embodiments, the detected variants within the one or more genes of interest may be used to determine whether a subject is a candidate for a PARP inhibitor or its biosimilars, including, but not limited to, olaparib (Lynparza®), niraparib (Zejula®), rucaparib (Rubraca®), talazoparib (Talzenna®).

In some embodiments, the detected variants within the one or more genes of interest may be used to determine whether a subject is a candidate for an AKT inhibitor or its biosimilars, including, but not limited to capivasertib (Truqap®), ipatasertib, afuresertib.

In some embodiments, the detected variants within the one or more genes of interest may be used to determine whether a subject is a candidate for a mTOR inhibitor or its biosimilars, including, but not limited to, sirolimus (Rapamune®), everilimus (Afinitor®, Zortress®, Afinitor Disperz®), temsirolumus (Torisel®), sapanisertib, vistusertib.

In some embodiments, the detected variants within the one or more genes of interest may be used to determine whether a subject is a candidate for a HER-2 targeted therapy or its biosimilars, including, but not limited to trastuzumab (Herceptin®), pertuzumab (Perjeta®).

### EXAMPLES

The following examples are put forth to provide those of ordinary skill in the art with a complete disclosure and description of how to make and use the invention of the present disclosure and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1

The example provided below is a nonlimiting example intended to illustrate one possible embodiment of the method or system described herein. It should not be construed as restricting the scope of said method or system, which are not limited to the specific details or configurations presented below. The method or system described herein encompass any modifications, variations, or alternatives that would be apparent to those skilled in the art, while maintaining the spirit and intended purposes of said method or system.

In a nonlimiting example, the apparatus, systems, and methods disclosed herein may be used to analyze the PTEN human gene. In such an example, the region of interest, including UTRs, exons, and introns, may cover 108,306 bp. A first set of probes was designed to provide a 1× tiling. A second set was designed to generate a 2× tiling when combined with the first, and a third set was designed to provide a 4× tiling when combined with the first two. The BLAT software was subsequently utilized for determining whether each of the designed probes aligns to multiple positions in the reference genomes. All probes with at least 15 additional BLAT hits with a score (number of bases that match and are not repeats) of at least 100 out of a maximum of 120 were discarded. Probes with at least 15 additional BLAT hits with a score of at least 75, but below 100, were retained but considered as part of the risky set.

In the nonlimiting example outlined above, five probes were completely discarded and 45 considered as risky, leaving 805 probes in the first set of probes, 771 in the second set, and 1538 in the third set. The designed probes were synthesized as biotinylated DNA and used alongside probes targeting other genes and regions, for library preparation from 24 test DNA samples. The SOPHIA GENETICS™ Universal Library Prep with CUMIN® barcodes commercial kit was used. The prepared libraries were sequenced on an Illumina NextSeq machine, in high-output mode. A total of 753,410,444 151-bp reads were generated, 99.3% of which were mapped to the reference genome.

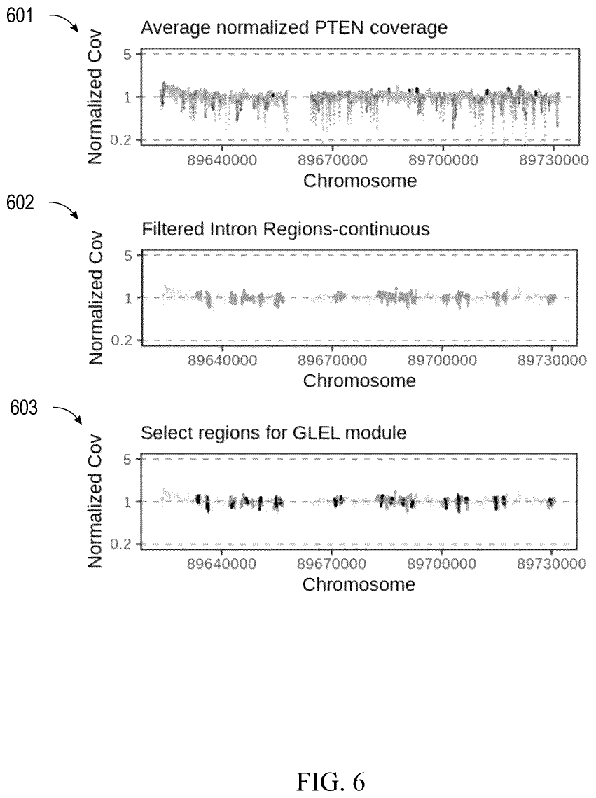

In continuance of the foregoing nonlimiting example, coverage depth was computed from the reads mapped to the PTEN region of interest and normalized within each sample by dividing by the sample-level average. Positions suitable for CNV analyses were then identified based on variation patterns across 20 of the samples. The coverage was obtained per 10-bp bins (See FIG. 6).

First, the standard deviation among samples of the normalized coverage was computed for each bin, and bins with a standard deviation above 0.15 were discarded. Second, the average normalized coverage across the 20 samples was computed for each bin, and bins with a coverage below 0.65 times the mean across bins were discarded, alongside 100 bp flanking the bin on each side. The remaining bins that could jointly create a continuous coverage across a minimum of 1000 bp were retained for analyses. For each continuous region, a single 500 bp sub-region defined in the center of each region was selected, representing virtual exons. A total of 19 virtual exons were delimited within the retained regions, which expanded the total analyzed sequence length by a six times factor, from ca. 1,800 bp based on genuine exons alone to ca. 11,300 bp across genuine and virtual exons.

Referring to FIG. 6, the identification of PTEN regions for intra-genic analysis of copy number variants may be illustrated by section 601. The normalized coverage averaged across samples is plotted across the region of interest of PTEN. Bins with a normalized coverage averaged among samples below 0.65 times the average of bins are in dark grey. The bins remaining after removing 10-bp bins with a standard deviation across the 20 samples above 0.15 and bins with a normalized coverage averaged among samples below 0.65 times the average of bins are shown in section 602. Among the bins in section 602, those providing continuous coverage are identified and highlighted in dark grey. In section 603, regions defined as virtual exons are moreover delimited in black within the continuous regions from section 602.

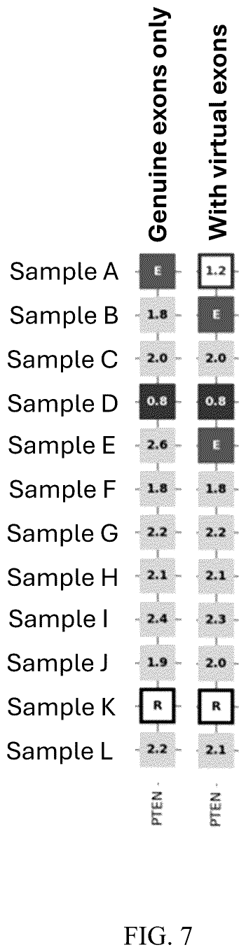

Coverage on the selected intronic regions, alongside the exonic regions of PTEN, may be employed as input for an exon-level CNV analysis, using the apparatus, systems, and methods disclosed herein. A bioinformatic pipeline implemented in the SOPHIA DDM™ genomic platform may be used, but the method disclosed herein may be compatible with a diversity of bioinformatic workflows. The results obtained with the virtual exons may be compared to results obtained without, demonstrating that the addition of virtual exons allows revealing exon-level rearrangements in several samples (e.g., Sample B and Sample E; FIG. 7).

Moving on to FIG. 7, the results from CNV analyses based only on genuine exons of PTEN or based on both genuine and virtual exons may be illustrated. Each square represents the CNV call for one sample, with the inferred number of copies indicated. Dark grey squares with a number indicate gene losses and open open squares with a number represent suspected gene losses. R=rejected sample based on quality, E=exon-level events.

As a nonlimiting example, the total number of reads covering the gene of interest were considered for structural variant analyses. A total of 11 fusion events were inferred from the analysis of discordant read pairs and reads spanning non-contiguous reference sequences, nine of which would not have been detected based solely on exon sequences. Five of these 11 fusions represent intra-PTEN rearrangements, while the others involve a potentially different fusion partner. The sample analysis was completed with the calling of four intronic indels, three of which were predicted to have splice consequences.

Results from the CNV analyses, structural rearrangements, and SNV and indel calling were combined to determine whether each of the samples has a PTEN loss of function. A loss of function was called if:
1. The gene, or some of its exons, were lost;
2. Exons were gained with structural rearrangements;
3. Structural rearrangements, such as deletions, gene fusions, or tandem duplications were detected; and
4. Large deletions of other variants changing the encoded proteins were detected.

Using these criteria, a PTEN loss of function was detected in 19 out of 20 samples. Based solely on exons, only the 9 with loss-of-function causing mutations within exons would have been identified. In other embodiments, any number or combination of the elements above may be utilized to determine whether a PTEN loss of function was detected.

Analyses of intra-genic structural variants for genes with few and/or short exons were previously hampered in capture-based NGS approaches by difficulties in efficiently capturing and sequencing intronic sequences. The apparatus, systems, and methods disclosed herein, provide an automatic and efficient way to design capture probes tiling the introns, and remove the barrier to allow the novel analytical approaches disclosed herein.

### Example 2

The example provided below is a nonlimiting example intended to illustrate one possible embodiment of the method or system described herein. It should not be construed as restricting the scope of said method or system, which are not limited to the specific details or configurations presented below. The method or system described herein encompass any modifications, variations, or alternatives that would be apparent to those skilled in the art, while maintaining the spirit and intended purposes of said method or system.

A total of 182 samples corresponding to subjects with prostate cancer were processed in an experiment designed to evaluate the usefulness of low-pass whole-genome sequencing (WGS) combined with hybrid-capture target enrichment for the holistic detection of loss-of-function in the gene PTEN.

DNA was extracted from biopsy tissue stored as formalin-fixed paraffin-embedded (FFPE) samples. Samples were processed in four batches. The first batch included 99 samples, which were sequenced using a whole-genome sequencing (WGS) approach. The second batch included the same 99 samples, but the samples were processed using a hybrid-capture target enrichment. The third batch included the 83 remaining samples, processed using a WGS approach. The fourth batch included the same 83 samples processed with the hybrid-capture target enrichment as in the second batch.

For the WGS sequencing, between 50 and 100 ng of each sample passing quality thresholds were processed using the commercial SOPHIA GENETICS™ Universal Library Prep with CUMIN™ Adaptors kit, using a mixture of manual and automated workflows. A total of 8 PCR cycles were used. The prepared libraries were sequenced on a NextSeq™ 2000 sequencer from Illumina™, producing about 16 million paired of 151-bp reads per sample.

For the hybrid-capture target enrichment, the same approach was used to produce WGS libraries. About 400 ng of each WGS library was then used as input for the capture as included in the commercial SOPHIA GENETICS™ Universal Library Prep with CUMIN™ Adaptors kit. The capture probes targeted the whole length of the PTEN gene and coding regions from 27 other genes; AKT1, ATM, BARD1, BRCA1, BRCA2, BRIP1, CCNE1, CDK12, CHEK1, CHEK2, ESR1, FANCA, FANCD2, FANCL, FGFR1, FGFR2, FGFR3, MRE11, NBN, PALB2, PIK3CA, PPP2R2A, RAD51B, RAD51C, RAD51D, RAD54L, and TP53. Between 12 and 24 WGS libraries with distinct sample indexes were included per pool for the capture. The hybrid capture was followed by 13 PCR cycles, and the capture libraries were sequenced on a NextSeq™ 2000 sequencer from Illumina™, producing about 10 million pairs of 151-bp reads per sample.

Reads were demultiplexed based on the sample indexes. Demultiplexed reads were cleaned to remove bad-quality sequences, adaptor sequences, and CUMIN™ barcodes.

Reads from the WGS libraries were used to calculate coverages. The genome was split in consecutive bins of 100,000 bp. The number of cleaned reads mapping to each bin was used to compute a coverage, which was then normalized per sample by dividing the coverage of each bin by the mean coverage among all bins for the sample. The normalized coverages were then multiplied by two, to obtain a mean of two copies, as expected in diploid organisms. Normalized genome-wide coverage profiles were manually reviewed for 99 samples from the first two batches. For each sample, the bin spanning the PTEN locus was visually inspected and classified relative to the sample's overall copy-number landscape rather than absolute depth. Regions with coverage comparable to the diploid baseline were labeled as ‘no PTEN deletion’. Regions showing a reduction in coverage consistent with single-copy loss, as judged by comparison to other putative heterozygous deletions within the same sample, were labeled as ‘heterozygous PTEN deletion’. Regions with more pronounced reduction, comparable to known biallelic losses elsewhere in the genome or below other putative heterozygous deletions, were labeled as ‘homozygous PTEN deletion’.

Reads from the capture and WGS libraries from each sample were then combined in silico to obtain a capture+WGS set of reads for each sample, mimicking a scenario where part of the WGS library is injected in the capture sequencing prior to sequencing [e.g. Pozzorini et al. Patent Pub. No. US 2022/0028481]. The genome was split in consecutive bins of 100,000 bp, and bins overlapping with target probes were excluded from follow-up analyses. The number of cleaned reads mapping to each gene was used to compute a coverage, which was then normalized per sample by dividing the coverage of each bin by the mean coverage among all bins for the sample. The normalized coverages were then multiplied by two, to obtain a mean of two copies, as expected in diploid organisms. The relative coverage of the region containing PTEN, which is excluded in these combined capture+WGS data because it overlaps with target probes, was calculated by dividing the mean normalized coverage of the ten 100-kbp bins on each side of PTEN by the mean normalized coverage of the other 100-kbp bins on the same chromosome (chromosome 10).

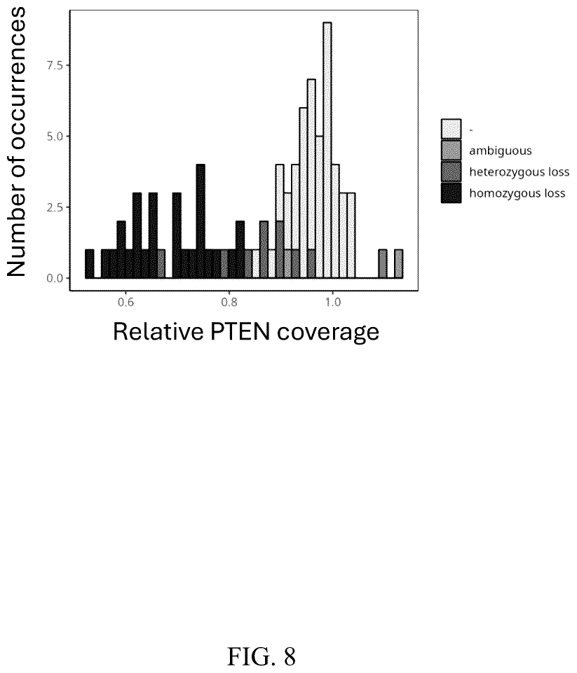

FIG. 8 shows the distribution of the relative coverage of the region containing PTEN among the 99 samples, with the grey scale indicating the classification based on WGS reads alone. Based on this distribution, a threshold of 0.87 was selected to distinguish samples with a likely homozygous deletion from other samples.

The 182 samples produced among all four batches were then processed using the same two approaches. The WGS coverages were manually inspected to infer a PTEN status, either as ‘homozygous PTEN deletion’ or ‘no homozygous PTEN deletion’ (which includes ‘heterozygous PTEN deletion’). The capture+WGS reads from the same 182 were then used to compute a relative PTEN coverage, as described above. These relative PTEN coverages were then used to automatically to classify samples as ‘homozygous PTEN deletion’ versus ‘no homozygous PTEN deletion’, using a threshold of 0.87. TABLE 1 shows the comparison between the manual WGS and threshold-based capture+WGS classifications. A total of 6 samples were considered ambiguous in the manual classification. Of the other samples, 44 were classified as ‘homozygous PTEN deletion’ in both the manual and threshold-based approaches and 122 were classified as ‘no homozygous PTEN deletion’ in both the manual and threshold-based approaches. The remaining 10 samples were classified differently in the manual and threshold-based approaches. The overall percent agreement (OPA) between the manual and threshold-based approaches was of 91.2%, the positive percent agreement (PPA) was of 93.6% and the negative percent agreement (NPA) was of 90.4%. The experiment therefore indicates that low-pass WGS coverage, obtained from the combined sequencing of capture and WGS libraries, enables the detection of homozygous PTEN deletions in a majority of cases.

TABLE 1
Comparison between Manual WGS and Threshold Based-Capture

| | | Manual Inspection | | |
|---|---|---|---|---|
| | | PTEN | No PTEN | |
| | | Loss | Loss | Ambiguous |
| Threshold-Based | PTEN Loss | 44 | 2 | 1 |
| Classification | No PTEN Loss | 8 | 122 | 5 |
| | Ambiguous | 0 | 0 | 0 |

The data produced were further used in a set of analyses to assess the power of combining low-pass whole genome data with higher coverage target enrichment data to elucidate loss of PTEN function events. Indeed, the capture-targeted regions were sequenced at a depth comprised between 5,000× and 15,000× for most samples, while the WGS coverage was below 2×.

Reads from the capture libraries were used to compute gene-level and exon-level CNVs from the PTEN gene using the instant method. In addition, reads from the capture libraries were used to detect structural variants. The normalized coverage obtained from WGS reads was then used to obtain genome-wide coverage plots, and the threshold-based analyses of the WGS reads described in Example 2 were conducted. For each sample, the PTEN status was evaluated by the joint consideration of the evidence provided by the different analyses of the capture and WGS reads.

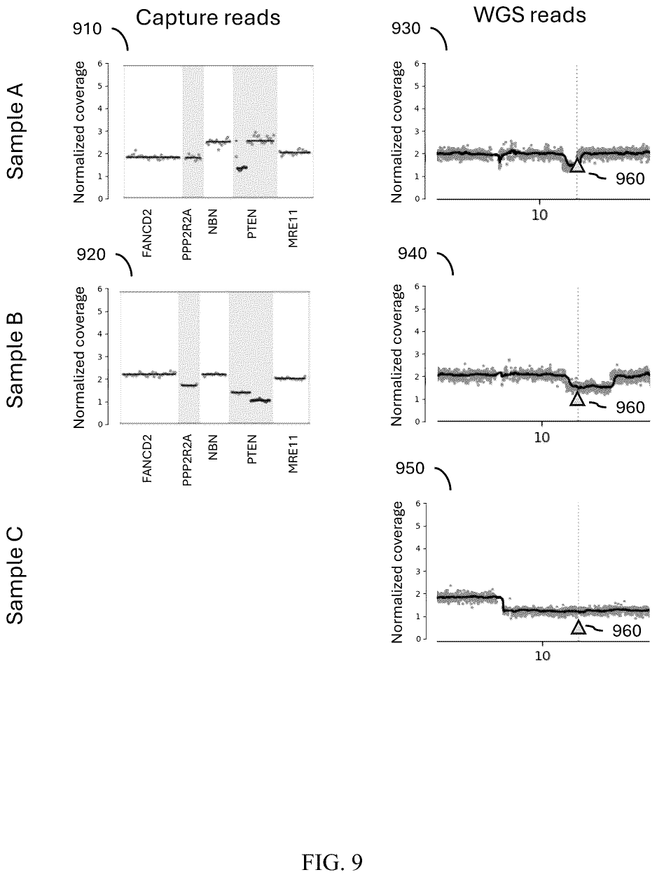

FIG. 9 provides examples of coverage patterns based on capture reads (left column) and WGS reads (right column), for three distinct samples (Sample A, Sample B, and Sample C).

For Sample A, the capture read coverage 910 suggested variation within the PTEN gene and the gene-level and exon-level CNV analysis identified a suspected exon loss, which was also supported by a breakpoint in the reads, indicating a structural variant. The WGS reads for the same Sample A 930 indicated a drop of coverage in the region including PTEN, represented by a grey triangle 960. Based on the different types of information, Sample A was therefore inferred to contain a heterozygous long deletion (~10 Mbp) spanning part of PTEN.

For Sample B, the capture read coverage 920 supports a deletion of part of PTEN, an event that was detected by the gene-level and exon-level CNV analysis as well as a breakpoint detected in the structural variant analysis. The analysis of WGS read coverage 940 revealed a drop of coverage along a long (~30 Mbp) region of chromosome 10 that includes PTEN. Within this region, the coverage of PTEN, indicated by a grey triangle 960, is further decreased. Taken together, the information provided by the capture reads and the WGS reads indicates that Sample B contains a heterozygous large deletion on one allele and a heterozygous intra-PTEN deletion on the other allele, leading to a homozygous loss of PTEN function.

For Sample C, no capture reads from PTEN were detected, indicating a homozygous loss of function (data not shown). The WGS read coverage 950 supports a heterozygous loss of one arm of chromosome 10. In addition, the coverage for PTEN, indicated with a grey triangle 960, is further decreased. Therefore, this sample has a heterozygous large-scale deletion on one allele coupled with a PTEN-specific deletion on the other allele, leading to a homozygous loss of function.

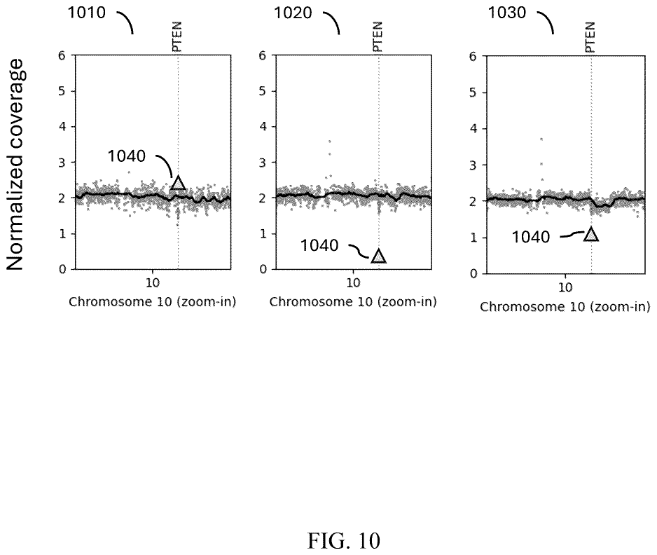

FIG. 10 provides examples additional of WGS coverage plots. In each case, the position of PTEN is highlighted with a grey triangle 1040. In the sample shown in the coverage plot 1010, PTEN has a coverage suggesting no deletion. In the sample shown in the coverage plot 1020, PTEN has a coverage near zero, suggesting a homozygous deletion. In the sample shown in the coverage plot 1030, PTEN has a coverage near 0.5, suggesting a heterozygous loss of function. Due to the restricted size of the deletion in the coverage plots 1010 and 1020, the event cannot be detected from the analysis of adjacent genomic bins. In such cases, analyses of coverage profiles based uniquely on WGS may be needed to elucidate the deletions.

These analyses combining insights from low-coverage WGS data and high-coverage target-enrichment data demonstrate the power of combining insights using the methods disclosed here for the detection of PTEN loss of function.

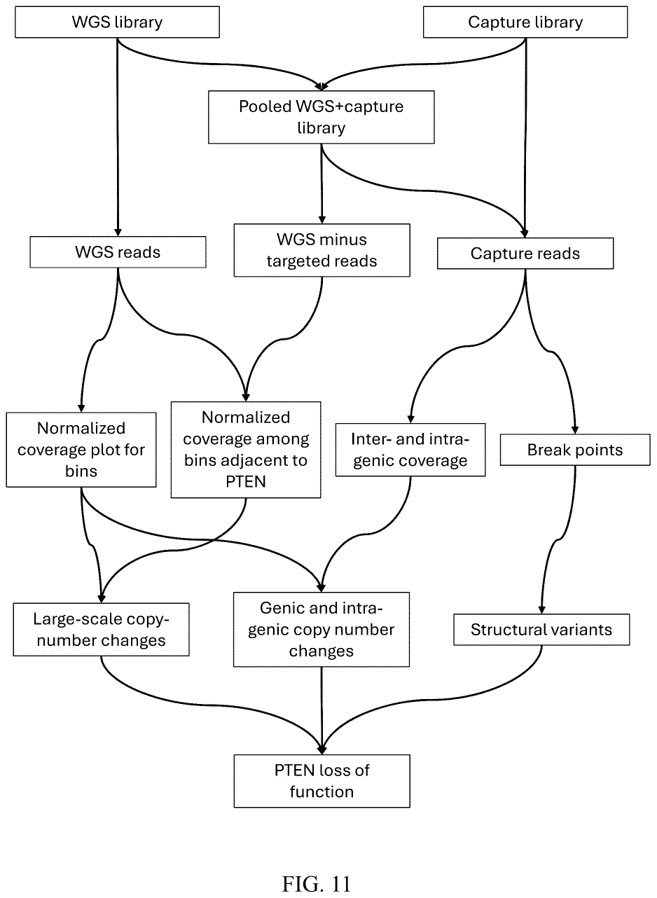

FIG. 11 illustrates how WGS and capture data can be combined to elucidate PTEN loss of function using the methods disclosed here. The workflow may start with the generation of a WGS library and a capture library. The two libraries may be sequenced independently, or the two libraries may be combined to produce a pooled WGS+capture library, which can then be sequenced. If the two libraries are sequenced independently, WGS reads and capture reads are produced.

If the two libraries were pooled before sequencing, then the reads can be sorted into capture reads, which overlap with the targeted genomic regions, and WGS reads minus targeted reads, which map to regions of the genome not targeted by the capture probes. Normalized coverage plots can be produced for all genomic bins from WGS reads. Because regions overlapping with the targeted regions are absent from the WGS minus targeted reads, only normalized coverage among genomic bins adjacent to genes targeted by the probes, such as PTEN, can be calculated.

The capture reads can be used to compute coverage per gene and among regions within each gene. In addition, the capture reads can be used to identify break points. The normalized coverage plot among bins, whether or not targeted regions are included, can be used to identify large-scale copy-number variants. In addition, the normalized coverage plot for bins including the targeted regions can be used to identify genic copy-number changes. The inter- and intra-genic coverage information can be used to identify genic and intra-genic copy number changes. Break points can be used to infer structural changes. Together, these different lines of evidence can be used to infer loss of function of PTEN. It will be apparent to those trained in the art that other markers, such as SNVs and indels, can be added to complete the information.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable sub-combination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

While the present invention has been described with reference to the specific embodiments thereof, it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method to detect loss of function variants within one or more genes of interest in a subject suffering from, or suspected to suffer from, a cancer, the method comprising the steps of:
(i) generating a whole-genome sequencing library from a plurality of DNA fragments derived from genomic DNA extracted from cells or cell-free DNA derived from bodily fluids;
(ii) using high-throughput sequencing to obtain sequencing reads from the sequencing library;
(iii) subjecting the sequencing reads to quality-based filters and mapping the resulting sequencing reads onto a reference genome;
(iv) detecting, from the sequencing reads, genetic variants that belong to at least two distinct categories, such as single nucleotide variants (SNVs), short insertions/deletions (indels), intra-genic copy number variants (CNV), large copy number variants (CNVs), and structural rearrangements; and
(v) combining information from at least two different types of genetic variants to determine whether the subject comprises loss of function of the one or more genes of interest.

2. The method of claim 1, wherein the whole-genome sequencing library is subjected to hybrid-capture target enrichment to produce a capture library prior to sequencing, and wherein capture probes cover coding and non-coding regions of the one or more genes of interest.

3. The method of claim 2, wherein part of the whole-genome sequencing library is mixed with the capture library before sequencing, and wherein the concentrations of the whole-genome sequencing library and capture library in the mix are selected to obtain a lower coverage for the whole-genome sequencing library in the sequencing reads.

4. The method of claim 3, wherein the reads originating from the capture library are used to detect intra-genic variants, such as SNVs, indels, CNVs, and structural variants.

5. The method of claim 4, wherein structural variants are identified based on the presence of discordant gene pairs or reads spanning sequences that are not contiguous within the reference genome.

6. The method of claim 5, wherein algorithms are deployed to detect discordant gene pairs or reads spanning non-contiguous reference sequences in numbers that allow confident inference.

7. The method of claim 4, wherein CNVs are inferred from coverage data, corresponding to the number of reads covering each position.

8. The method of claim 7, wherein inferring the CNVs is performed using a hidden Markov model (HMM), which assigns copy numbers based on observed coverage data and an underlying model.

9. The method of claim 8, wherein CNVs are inferred independently, at the whole-gene level and at the exon level, and wherein the whole-gene level and exon level CNV estimates are combined to report CNVs within genes in addition to CNV of the whole gene.

10. The method of claim 9, wherein the exon-level CNVs are inferred based on the coverage of genuine exons and virtual exons defined within the introns.

11. The method of claim 8, wherein estimates of gene-level CNVs and exon-level CNVs are combined with read-based estimates of structural variants to infer rearrangements, by reporting high-confidence structural variants when they are detected with both methods.

12. The method of claim 3, wherein the reads originating from the whole-genome sequencing library are used to calculate coverage among genomic bins and infer CNVs among genomic regions spanning at least 10,000 bp.

13. The method of claim 12, wherein the inferring copy number is performed by comparing the coverage of one or more selected genomic bins of interest to other genomic bins along the same chromosome.

14. The method of claim 1, wherein a loss of function is determined if:
(i) any of a gene loss, an exon loss, a gene fusion, a SNV causing a premature STOP codon, or an indel causing a reading frame shift is detected;
(ii) a SNV that causes an amino acid change that is predicted to be pathogenic is detected; or
(iii) a combination thereof.

15. The method of claim 14, wherein a homozygous loss of function is inferred if two loss-of-function causing genetic variants can be assigned to distinct alleles.

16. The method of claim 2, wherein the capture probes are designed based on a reference genome, so that they cover most of the gene sequence, including one or more of exons, introns, and UTRs.

17. The method of claim 16, wherein the set of capture probes represent a combination of different sets of probes.

18. The method of claim 1, wherein the one or more genes of interest include the gene PTEN.

19. A computer-implemented software to identify loss-of-function variants from high-throughput sequencing data using the method of claim 1.

20. A set of reagents used to produce sequencing libraries compatible with the method of claim 1.
